(19) United States
(12) Patent Application Publication
(10) Pub. No.: US 2013/0065789 A1
(43) Pub. Date: Mar. 14, 2013

(54) COMPOSITIONS AND METHODS FOR CLASSIFYING LUNG CANCER AND PROGNOSING LUNG CANCER SURVIVAL

(71) Applicants: University Health Network, Toronto (CA); British Columbia Cancer Agency Branch, Vancouver (CA)

(72) Inventors: Ming-Sound Tsao, Toronto (CA); Sarit Aviel-Ronen, Shohan (IL); Wan Lam, Vancouver (CA); Bradley Coe, Vancouver (CA); Chang-Qi Zhu, Thornhill (CA); Igor Jurisica, Toronto (CA)

(73) Assignees: BRITISH COLUMBIA CANCER AGENCY BRANCH, Vancouver, BC (CA); UNIVERSITY HEALTH NETWORK, Toronto, ON (CA)

(21) Appl. No.: 13/671,912

(22) Filed: Nov. 8, 2012

Related U.S. Application Data
(63) Continuation of application No. 12/996,064, filed on Feb. 28, 2011.
(60) Provisional application No. 61/059,085, filed on Jun. 5, 2008.

(51) Int. Cl. C12Q 1/68; C40B 40/06

(52) U.S. Cl. CPC C12Q 1/6837; C12Q 1/686; C40B 40/06
USPC 506/9; 435/6.12; 435/7.1; 436/501; 435/6.11; 506/16

(57) ABSTRACT

The application provides methods of prognosmg, diagnosing, screening and classifying lung cancer patients into poor survival groups or good survival groups. A number of altered genomic regions have been identified that distinguish subtype of lung adenocarcinoma (ADC), specifically between bronchioloalveolar carcinoma (BAC) and invasive ADC with BAC features (AWBF), and genes and biomarkers whose expression are altered in individuals with pulmonary ADC according to different survival outcomes. The amplification and/or deletion of these genomic regions, and/or the biomarker expression profiles can be used to classify patients with ADC into a BAC group with excellent survival outcome, or an invasive ADC with BAC features group with higher risk of developing metastatic recurrence and poorer survival outcome. The application also includes kits for use in the methods of the application.

### FIELD

The application relates to compositions and methods for classifying, diagnosing and prognosing lung cancer, particularly pulmonary adenocarcinoma (ADC).

### BACKGROUND

Lung adenocarcinoma (ADC) accounts for approximately 35% of all lung cancers and has an overall 5-year survival of 17% (1). The recent World Health Organization (WHO) classification recognized a particular subtype, bronchioloalveolar carcinoma (BAC), for its non-invasive features and excellent prognosis (2). BAC has a distinct histological pattern with tumor cells growing along pre-existing alveolar framework, without evidence of stromal, pleural or vascular invasion. Yet, some invasive ADC, classified as mixed type, may have components or large areas of BAC-like pattern. Multi-stage development of adenocarcinoma putatively involves progression from atypical adenomatous hyperplasia (AAH) through BAC to invasive ADC with BAC features (AWBF) (3-5). Mice that express oncogenic KRAS develop histological changes that range from mild hyperplasia/dysplasia analogous to atypical adenomatous hyperplasia to alveolar adenomas and ultimately displayed overt ADC (6, 7).

The identification of genes/proteins that may distinguish BAC from AWBF, and are predictors of ADC with poor prognosis, would be useful for the establishment of novel molecular pathological classification of lung adenocarcinoma.

### SUMMARY

Disclosed herein are genomic and expression profiles of different subtypes of lung adenocarcinoma (ADC). A number of altered genomic regions have been identified that distinguish subtype of lung adenocarcinoma (ADC), specifically between bronchioloalveolar carcinoma (BAC) and invasive ADC with BAC features (AWBF), and genes or biomarkers whose expression are altered in individuals with pulmonary ADC according to different survival outcomes. The amplification and/or deletion of these genomic regions, and/or the biomarker expression profiles can be used to classify patients with ADC into a bronchioloalveolar carcinoma (BAC) group with excellent survival outcome, or an invasive ADC with BAC features (AWDF) group with higher risk of developing metastatic recurrence and poorer survival outcome.

Accordingly, one aspect of the application provides a method of classifying or prognosing a subject with lung ADC, comprising the steps:

(a) determining a genomic profile in a test sample from the subject,

(b) comparing the genomic profile with a control;

wherein a difference or a similarity in the genomic profile between the control and the test sample is used to classify the subject with lung ADC into a BAC or an invasive ADC group, and/or prognose the subject as having poor survival or a good survival.

In an embodiment, the control comprises a reference genomic profile associated of a disease free and/or non-tumor sample, and a difference in the genomic profile between the control and the test sample is indicative of invasive ADC. In an embodiment, the control comprises a threshold level, for example a gene copy number fold change threshold, above which the subject is classified as belonging to an invasive ADC group, is diagnosed as having invasive ADC such as AWBF, and/or is prognosed as having poor survival.

In an embodiment, the control comprises a reference genomic profile associated with invasive ADC and/or poor survival, and a similarity in the genomic profile between the control and the test sample is indicative that the subject with lung adenocarcinoma is classified as having invasive ADC, and/or is prognosed as having a poor survival.

In another embodiment, the control is a reference genomic profile corresponding to a subject with BAC and/or good survival, and a similarity in the genomic profile between the control and the test sample is indicative that the subject is classified as having BAC and/or prognosed as having good survival.

The above described genome alterations are reflected in a number of genes or biomarkers which are altered in their copy number and/or differentially expressed in individuals with pulmonary ADC. Detecting the gene copy number e.g. the amplification and/or deletion of these biomarkers and/or their differential expression can be used to classify patients with ADC into a BAC group, or an invasive ADC group, to diagnose the subject as having BAC or invasive ADC, such as AWBF, and/or to prognose the subject as having a good prognosis or a poor prognosis.

The amplification and/or deletion and/or differential expression of these biomarkers, for example the biomarkers in Tables 3 and 4, as well as in Table 13 can also be used to prognose patients with ADC into a poor survival group or a good survival group.

Accordingly, in an aspect, the application provides methods of classifying a subject with ADC into a BAC group with an excellent survival outcome, or an invasive ADC with higher risk of developing metastatic recurrence and a poor survival outcome, using biomarker gene copy number and/or biomarker expression product levels of one or more of the biomarkers described herein. The expression products can include RNA products and polypeptide products of the biomarkers.

An embodiment provides a method of classifying a subject with lung adenocarcinoma, comprising the steps:

(a) determining the gene copy number and/or the expression level of one or more biomarkers in a test sample from the subject, wherein each biomarker corresponds to a gene in Table 1, 2, 3, 4, and/or 13,

(b) comparing the gene copy number and/or the expression level of the one or more biomarkers with a control,

wherein a difference in the gene copy number and/or the expression level of the one or more biomarkers between the control and the test sample is used to classify the subject with lung adenocarcinoma into a BAC or an invasive ADC group.

Another aspect relates to diagnosing a subtype of lung adenocarcinoma in a subject comprising the steps:

(a) determining the expression level of one or more biomarkers in a test sample from the subject, wherein each biomarker corresponds to a gene in Table 1, 2, 3 and/or 4;

(b) comparing the expression of the one or more biomarkers with a control,

wherein a difference or a similarity in the expression of the one or more biomarkers between the test sample and the control is used to diagnose the subject has having BAC or invasive ADC.

Another embodiment provides a method for diagnosing a subtype of lung adenocarcinoma in a subject comprising the steps:
a) obtaining a subject biomarker expression profile in a sample of the subject;
b) obtaining one or more biomarker reference expression profiles associated with a subtype of lung adenocarcinoma, wherein the subject biomarker expression profile and the biomarker reference profile each has a plurality of values, each value representing the expression level of a biomarker, wherein each biomarker corresponds to a gene in Table 1, 2, 3 and/or 4; and
c) selecting the biomarker reference expression profile most similar to the subject biomarker expression profile, to thereby diagnose the subject as having BAC or invasive ADC.

Another embodiment provides a method of prognosing a subject with lung adenocarcinoma, comprising the steps:

(a) determining a gene copy number and/or an expression level of one or more biomarkers in a test sample from the subject, wherein each biomarker corresponds to a gene in Table 1 and/or 3.

(b) comparing the gene copy number and/or the expression level of the one or more biomarkers with a control,

wherein a difference in gene copy number and/or expression level of the one or more biomarkers between control and the test sample is used to prognose the subject with lung adenocarcinoma into a poor survival group or a good survival group.

In another embodiment, the difference in gene copy number comprises a gene amplification of the one or more biomarkers. In another embodiment, the difference in gene copy number comprises a gene deletion of the one or more biomarkers. In certain embodiments, the difference in the gene copy number and/or expression level comprises amplification and/or increased expression of one or more of the genes in Table 1, 3 and/or 13 compared to a control. In other embodiments the gene copy number comprises deletions in and/or decreased expression of one or more genes in Table 2 and/or 4 compared to a control. In other embodiments, the gene copy number and/or expression level comprises amplification or increased expression of one or more genes in Table 1, 3 and/or 13 and deletions or decreased expression in one or more genes in Table 2 and/or 4 compared to a control. In certain embodiments, the control is a gene copy number of a gene in Table 1 or 2 from a disease free, and/or non-tumor sample.

In another embodiment, the gene amplification and/or an increased expression level of the one or more biomarkers of Table 1, 3 and/or 13 and/or a gene deletion and/or a decreased expression level of the one or more biomarkers of Table 2 and/or 4 between the control and the test sample is used to classify the subject with lung adenocarcinoma into a BAC or an invasive ADC group and/or prognose the subject with lung ADC into a poor survival group or a good survival group.

In another embodiment, a gene amplification and/or an increased expression level of the one or more biomarkers of Table 1 3 and/or 13 and/or a gene deletion and/or a decreased expression level of the one or more biomarkers of Table 2 and/or 4 between the control and the test sample is indicative that the subject with lung adenocarcinoma has invasive ADC and/or poor survival.

The one or more biomarkers whose level of expression is determined, is in one embodiment selected from Table 3, 4 and/or 13.

The prognoses, diagnoses and classifying methods of the application can be used to select treatment. For example, the methods can be used to select or identify what type of treatment is indicated.

Another aspect of the application provides compositions useful for use with the methods described herein. In an embodiment, the compositions comprise one or more primers for detecting a biomarker described herein.

The application also provides for kits used to classify, diagnose and/or prognose a subject with ADC into a BAC with good survival outcome or an invasive ADC with poorer survival outcome that includes detection agents that can detect the gene copy number or expression level of one or more of the biomarkers disclosed herein.

Other features and advantages of the present application will become apparent from the following detailed description. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

The application will now be described in relation to the drawings in which:

FIG. 1: BAC compared to AWBF by histology, frequency scoring and threshold filtering. A1—Bronchioloalveolar carcinoma showing typical growth pattern of tumoral cells along the pre-existing alveolar scaffold without evidence of invasion (HE X100). A2—AWBF has both BAC-like and invasive areas (HE X16). Inset: high power of the invasive component (HE X100).

FIG. 2: TERT validation by qPCR and FISH. A—TERT content measured by array CGH and qPCR on genomic DNA (relative to normal control), and FISH (mean gene copy number per nucleus) show high correlation between the different methods. The black lines connecting copy number of TERT and 5q are drawn to highlight the difference in copy number between the two probes. The boxes mark the AWBF sampled in invasive area (T46B, T43B, T41B, and T44B). For samples T41, T44 and T46, “A” represents the BAC-like area and “B” the invasive area of AWBF. B—FISH performed on AWBF in BAC area (sample number T195) using the dual-color FISH probe mix that contains the hTERT locus (5p15, non-circled signal) and the control D5S89 probe (5q31, circled signal). Gain of TERT is reflected by the increased number of green compared to red foci.

FIG. 3: PDCD6 validation and markers of poor prognosis. A—qPCR performed on genomic DNA showed statistically significant differences in PDCD6 gene copy number between BAC and AWBF (p=0.01), confirming the array CGH analysis that identified its amplification. B—qPCR performed on 10 paired cDNAs of ADC-normal samples. PDCD6 was significantly overexpressed in tumor compared to normal lung tissue (p<0.01), with a mean 3-fold higher expression. C—Multivariate analysis adjusting for stage, histology, and differentiation that relied on qPCR of cDNA from 85 NSCLC samples, found that PDCD6 was an independent poor prognostic factor for overall survival in stage I-II ADC patients. D, E, F—Kaplan Meier survival curves of SERPINE1, GNB2 and ST13, respectively, based on gene expression data from the ‘Duke’ database. Expression data was dichotomized at the median.

### DETAILED DESCRIPTION

The application relates to genomic alterations, gene copy number variations and differential biomarker expression levels and profiles in subjects with lung adenomacarcinoma or NSCLC which are associated with a classification, diagnosis and/or prognosis and provides methods, compositions, detection agents and kits for classifying, diagnosing or prognosing a subject with lung adenocarcinoma or NSCLC.

### I. DEFINITIONS

The term “lung adenocarcinoma” and/or “lung ADC” and/or “pulmonary ADC” as used herein refer to a type of lung cancer and comprises various subtypes including bronchioloalveolar carcinoma (BAC) which is non invasive and/or includes focal invasion and has good prognosis (2) and invasive ADC including mixed type, which can have areas with BAC like pattern and is referred to as invasive ADC with BAC features (AWBF).

The term “invasive ADC” as used herein refers to lung ADC that is invasive, with or without areas of BAC like pattern and includes AWBF. Subjects with invasive ADC can have poor prognosis or good prognosis. Expression levels of biomarkers corresponding to genes, for example one or more genes listed in Table 3 and/or 4, are useful for differentiating more indolent from aggressive forms of invasive ADC, which have good prognosis.

The term “bronchioloalveolar carcinoma” or “BAC” as used herein refers to a subtype of lung ADC which is non-invasive and/or includes focal invasion (i.e. BAC with focal invasion) and has good prognosis.

The term “non-small cell lung cancer” as used herein refers to primary lung cancer that is distinguished from small cell lung cancer and that is composed of multiple different types, including adenocarcinoma, squamous cell carcinoma, large cell carcinoma and other less frequent types.

The term “biomarker” or “marker” as used herein refers to a gene that is altered in its gene copy number and/or is differentially expressed, in individuals with ADC according to ADC classification, diagnosis and/or prognosis. The biomarkers are diagnostic, useful for classifying subjects and predictive of different survival outcomes. For example the term “biomarkers” includes one or more of the genes listed in Table 1, 2, 3, 4 and/or 13 such as EPO, SLC25A17, POP7, PDCD6, SERPINE1, GNB2, and ST13.

As used herein, the term “control” refers to a specific value or dataset e.g., control expression level, control gene copy number, reference expression profile or reference genomic profile, derived from a known subject class e.g., from a sample of a disease free subject; a subject with BAC and/or a subject with invasive ADC, for example AWBF, and/or normal tissue such as tumor adjacent non-neoplastic tissue, that can be compared to and used to classify, diagnose or prognose the value or dataset derived from a test sample, e.g., expression level, gene copy number, expression profile or genomic profile, obtained from the test sample. For example, the control can be normal tissue. Normal tissue with respect to genomic profile refers to a single genomic copy on each of the two alleles. For example, the control can be derived from samples from a group of subjects known to have lung ADC and/or good survival outcome or known to have lung ADC and/or have poor survival outcome. In another example, the dataset can be derived from a sample from a group of subjects known to have BAC, or a group of subjects known to have invasive ADC and/or AWBF. The control is optionally a value such as a threshold level. For example, it is shown herein that for a desired or particular sensitivity and/or specificity the control can be a threshold level as indicated for example in Table 13. Accordingly for example, where the control is a threshold level for a particular biomarker (e.g. gene copy number fold change threshold), samples that have a gene copy number above the threshold value are classified as belonging to an invasive ADC group such as AWBF, diagnosed as having AWBF and/or prognosed to have poor survival and/or tumor progression. A person skilled in the art will recognize that different threshold levels can be used depending upon the desired specificity and sensitivity. Optionally one or more controls can be used, for example an internal control can be used with or without comparison to a control sample, such as a tissue sample. With respect to genomic alterations e.g. gains and losses, the control can for example also refer to an internal control e.g the copy number of a nonaltered region of the chromosome or a different chromosome e.g a chromosome with minimal variance in lung cancer subjects, for example a chromosome not herein or previously identified as associated with prognosis. Such methods wherein an internal control is useful include for example quantitative polymerase chain reaction (PCR) or fluorescent in situ hybridization (FISH). Optionally, the copy number can be compared to the centromere for example when using FISH. Typically a normal or control genomic profile refers to a single genomic copy on each of the two alleles. For example in the array-CGH, the control is a normal reference genomic DNA that is assumed to have 2 copies of each gene. In other examples, the control is optionally a positive control or a negative control, for example for quantitative PCR and/or FISH methods, for example included in quantitative PCR and/or FISH based kits. Based on the teachings herein and knowledge in the field, a person skilled in the art would readily be able to identify suitable controls for the methods described herein. Similarly, an internal control can be used to normalize for expression levels, for example a house keeping gene can be used in a quantitative RT-PCR protocol.

The term “gene copy number fold change threshold” refers to a value that identifies for a particular sensitivity and specificity a copy number that distinguishes between two classes, diagnoses and/or prognoses and which can be used to classify, diagnose or prognose tests samples, e.g the gene copy number in a test sample is compared to the gene copy number fold threshold (e.g. as a control) above which a subject is classified as belonging for example to a class with poor prognosis, diagnosed as having for example AWBF, and/or prognosed as having poor survival. For example, Table 13 indicates that the biomarker PPA1 has a gene copy number fold change threshold of 1.2 for a specificity of 91.7%, and a sensitivity of 53.3%. Test samples having a copy number of PPA1 above 1.2 are for example classified has having a poor prognosis, diagnosed as having AWBF and/or prognosed as having poor survival. For example, the gene copy number fold change threshold can be determined as described in Example 3.

The term “disease free subject” refers to a subject that is free of lung adenocarcinoma.

The term “reference profile” as used herein refers to a reference expression profile, a reference genomic profile, and/or a reference gene copy number profile according to the context.

A “reference expression profile” as used herein refers to the expression signature of a subset of biomarkers which correspond to genes associated with a clinical classification, diagnosis and/or outcome in a lung adenocarcinoma patient and/or ADC disease free subject. The reference expression profile can comprise a plurality of values, each value representing the expression level of a biomarker in a control, wherein each biomarker corresponds to a gene in Table 1, 2, 3, 4 and/or 13. For example, with respect to classification, the reference expression profile can refer to the expression signature of a subset of biomarkers listed in Table 1 and/or 2 which are differentially expressed in BAC and invasive ADC groups. With respect to prognosis, for example, the reference expression profile can refer to the expression signature of a subset of biomarkers listed in Table 3 and/or 4, which are differentially expressed in patients in a poor survival group or a good survival group. The reference expression profile is optionally derived de novo from a control and/or can be a standard value previously derived from one or more known control samples. For example, the reference expression profile can be a predetermined value for each biomarker or set of biomarkers derived from ADC patients whose biomarker expression values and/or survival outcomes are known. Using values from known samples allows one to develop an algorithm for classifying new patient samples into good and poor prognostic groups as described in the Example. The reference expression profile is identified using one or more samples comprising tumor wherein the expression is similar between related samples defining a class e.g., BAC or invasive ADC and/or an outcome group such as poor survival or good survival and is different to unrelated samples defining a different class and/or outcome group such that the reference expression profile is associated with a particular clinical class or outcome. The reference expression profile is accordingly a reference profile or reference signature of the expression of a subset of genes, for example the genes in Table 1, 2, 3, 4 and/or 13, to which the subject expression levels of the corresponding genes in a test sample can be compared in methods for determining or predicting clinical class or outcome. A person skilled in the art will recognize that a variety of methods can be used to determine a reference expression profile or an expression signature. For example, a reference expression profile or an expression signature can be determined by amplification of polynucleotides.

The term “expression level” as used herein refers to the absolute or relative amount of the transcription and/or translation product of a biomarker described herein and includes RNA and polypeptide products. A person skilled in the art will be familiar with a number of methods that can be used to determine RNA transcription levels, such as qRT-PCR and/or polypeptide levels such as immunohistochemistry.

A “reference gene copy number profile” as used herein refers to the gene copy number of a subset of genes listed in Tables 1, 2, 3, 4 and/or 13 associated with ADC classification, diagnosis and/or clinical outcome in a lung adenocarcinoma patient and/or ADC disease free subject. The reference gene copy number profile comprises a plurality of values, each value representing the copy number of a gene in Tables 1, 2, 3, 4 and/or 13. The reference gene copy number profile is identified using for example normal human tissue and/or cells and/or tissue and/or cells from lung ADC subtypes. Normal tissue and/or cells includes for example, tumor adjacent non-neoplastic tissue and/or cells and/or tissue and/or cells from a lung cancer disease free subject. The reference gene copy number profile is accordingly a reference signature of the copy number of a subset of genes in Tables 1, 2, 3, 4 and/or 13, to which the subject gene copy number of the corresponding genes in a test sample are compared.

The term “genomic profile” as used herein refers to the genomic structural signature of an individual's genome. A number of variations and alterations referred to as copy number variations, have been characterized including amplifications and deletions (e.g. losses and gains), a subset of which are associated with disease subtype and/or prognosis. The alterations can comprise small and large amplifications and/or deletions which can occur through out the genome.

The term “loss” or “gain” refers with respect to a genomic profile refers to a change in copy number, for example the loss can be on the plus strand or the minus strand and can involve loss of one or both alleles. Similarly, a “gain” can for example be a gain on the plus strand or the minus strand and can involve gain on one or both alleles. The gain can additionally be the gain of 1 or more copies.

The phrase “determining a genomic profile” as used herein refers to detecting the presence, frequency, variability and/or length of one or more genomic alterations including amplifications and deletions which may or may not comprise alterations in the nucleic acid sequence of genes e.g., can comprise alterations in the intergenic regions of the genome. Genomic alterations comprising amplifications and deletions in genes comprise those listed in Tables 1, 2, 3, 4 and/or 13. A person skilled in the art will appreciate that a number of methods can be used to determine a genomic profile, including for example fluorescence and other non-fluorescent types of in situ hybridization (FISH, CISH or others), amplification methods such as quantitative PCR (qPCR), multiplex PCR including for example multiplex ligation dependent probe amplification (MLPA) as well as array CGH.

Amplification of polynucleotides utilizes methods such as the polymerase chain reaction (PCR), including for example quantitative PCR, multiplex PCR and multiplex ligation dependent probe amplification (MLPA), ligation amplification (or ligase chain reaction, LCR) and amplification methods based on the use of Q-beta replicase. These methods are well known and widely practiced in the art. Reagents and hardware for conducting PCR are commercially available. Primers useful to amplify specific sequences from selected genomic regions are preferably complementary to, and hybridize specifically to sequences flanking the target genomic regions.

The term “reference genomic profile” as used herein refers a genomic signature comprising genomic alterations, associated with classification and/or clinical outcome in lung ADC patients and/or an ADC disease free subject. The reference genomic profile comprises a plurality of values, each value representing a change in a genomic region. The reference genomic profile is for example derived from normal human tissues and/or cells. The reference genomic profile is accordingly for example, normal genomic copy to which a subject genomic profile is compared for classifying the tumor, diagnosing a clinical subtype or determining or predicting clinical outcome.

The terms “complementary” or “complementarity”, as used herein, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some nucleotides or portions of the nucleotide sequences of the nucleic acids bind, or it may be complete when total complementarity exists between the single stranded molecules. The degree of complementarity between nucleic acid strands has significant effects on the efficiency and strength of hybridization between nucleic acid strands.

The term “similar” or “similarity” as used herein with respect to a reference profile refers to similarly in both the identity and quantum of change in expression level of a biomarker, genomic alteration, or gene copy number variation compared to a control where the control is for example derived from a normal cell and/or tissue or has a known diagnosis, or outcome class such as poor survival or good survival.

The term “similarity in expression” as used herein means that there is no or little difference, for example no statistical difference, in the level of expression of the biomarkers between the test sample and the control and/or between classes, diagnostic groups, and good and poor prognosis groups defined by biomarker expression levels.

The term “most similar” in the context of a reference profile refers to a reference profile that is associated with a class, diagnosis or clinical outcome that shows the greatest number of identities and/or degree of changes with the subject profile.

The term “differentially expressed” or “differential expression” as used herein refers to biomarkers described herein that are expressed at one level in an ADC class, diagnostic or prognostic group and expressed at another level in a control. The differential expression can be assayed by measuring the level of expression of the transcription and/or translation products of the biomarkers, such as the difference in level of messenger RNA transcript expressed or polypeptide expressed in a test sample and a control. The difference can be statistically significant.

The term “difference in the level of expression” refers to an increase or decrease in the measurable expression level of a given biomarker expression product as measured by the amount of messenger RNA transcript and/or the amount of polypeptide in a sample as compared with the measurable expression level of a given biomarker in a control. In an embodiment, the differential expression can be compared using the ratio of the level of expression of a given biomarker or biomarkers as compared with the expression level of the given biomarker or biomarkers of a control, wherein the ratio is not equal to 1.0. For example, an RNA or polypeptide is differentially expressed if the ratio of the level of expression in a first sample as compared with a second sample is greater than or less than 1.0. For example, a ratio of greater than 1, 1.2, 1.5, 1.7, 2, 3, 4, 5, 10, 15, 20 or more, or a ratio less than 1, 0.8, 0.6, 0.4, 0.2, 0.1, 0.05, 0.001 or less. In another example, the differential expression is measured using p-value. For instance, when using p-value, a biomarker is identified as having a “difference in the level of expression” as between a first sample and a second sample when the p-value is less than 0.1, preferably less than 0.05, more preferably less than 0.01, even more preferably less than 0.005, the most preferably less than 0.001.

The term “prognosis” as used herein refers to a clinical outcome group such as a poor survival group or a good survival group which is reflected by a reference profile such as a reference expression profile, or a reference gene copy number profile, or reflected by an expression level of one or more biomarkers disclosed herein. It can also be reflected by genomic alterations. The prognosis provides an indication of disease progression and includes an indication of likelihood of recurrence, metastasis, death due to disease, tumor subtype or tumor type. The clinical outcome class includes a good survival group and a poor survival group.

The term “classifying” as used herein means identifying and/or diagnosing the clinical subtype of lung ADC. For example, lung ADC includes subtypes bronchioloalveolar carcinoma (BAC) which is non invasive and/or has focal invasions and has good prognosis (2) and invasive ADC including mixed type, which can have areas with BAC like pattern and is referred to as invasive ADC with BAC features (AWBF), which can have poor prognosis. “Classifying” can therefore refer to a method or process of determining whether an individual with ADC has BAC or invasive ADC and/or AWBF.

The term “diagnosing” as used herein means identifying an illness or subtype such as BAC or invasive ADC.

The term “prognosing” as used herein means predicting the course of disease or identifying the clinical outcome group a subject belongs to according to the subject's similarity to a control and/or a reference profile and/or biomarker expression level associated with the prognosis. For example, prognosing comprises a method or process of determining whether an individual with ADC has a good or poor survival outcome, or grouping an individual with ADC into a good survival group or a poor survival group. The term “good survival” as used herein refers to an increased chance of survival as compared to patients in the “poor survival” group. For example, the biomarkers of the application can prognose patients into a “good survival group” for example which includes subjects with BAC and/or less aggressive invasive ADC. These patients are at less risk of death 5 years after surgery. The good survival group comprises subjects having a 5 year survival rate of about 80% or more.

The term “poor survival” as used herein refers to an increased risk of death as compared to patients in the “good survival” group. For example, biomarkers or genes of the application can prognose patients into a “poor survival group” which include for example patients with more aggressive forms of invasive ADC and/or subjects with mixed type adenocarcinoma with BAC features (AWBF). These patients are at greater risk of death within 5 years from surgery. For example the poor survival group comprises subjects having a 5 year survival rate of less than about 80%.

As used herein, “treatment” is an indicated approach for obtaining beneficial or desired results, including clinical results, for example an indicated approach for lung ADC subtypes. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, prolonging survival as compared to expected survival if not receiving treatment and remission (whether partial or total), whether detectable or undetectable. For example surgery or chemotherapy are indicated treatments for subjects with invasive ADC while BAC patients may be treated with limited resection or non-invasive or minimally invasive procedures.

“Palliating” a disease or disorder means that the extent and/or undesirable clinical manifestations of a disorder or a disease state are lessened and/or time course of the progression is slowed or lengthened, as compared to not treating the disorder.

The phrase “selecting a treatment” as used herein refers to selecting any indicated treatment that is useful for obtaining beneficial results such as prolonging survival and/or palliation.

The term “subject” as used herein refers to any member of the animal kingdom, preferably a human being. A “subject with ADC” as used herein includes a subject that has ADC or that is suspected of having ADC.

The term “test sample” as used herein refers to any fluid, cell or tissue sample from a subject which can be assayed for biomarker expression products and/or a reference expression profile, e.g. genes differentially expressed in subjects with ADC according to survival outcome and/or for which a genomic profile can be determined and includes without limitation tumor tissue and/or cells, derived from, for example, lung biopsy, for example obtained by bronchoscopy, needle aspiration, thoracentesis and/or thoracotomy, and/or derived from cells found in sputum.

The phrase “determining the expression level of biomarkers” as used herein refers to determining a level, including a relative level, or quantifying RNA transcripts and/or polypeptides expressed by the biomarkers. The term “RNA” includes mRNA transcripts, and/or specific spliced variants of mRNA. The term “RNA product of the biomarker” as used herein refers to RNA transcripts transcribed from the biomarkers and/or specific spliced variants. In the case of “polypeptide”, it refers to polypeptides translated from the RNA transcripts transcribed from the biomarkers. The term “polypeptide product of the biomarker” refers to polypeptide translated from RNA products of the biomarkers.

The term “nucleic acid” includes DNA and RNA and can be either double stranded or single stranded.

The term “hybridize” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed.

The term “detection agent” as used herein refers to any molecule or compound that is useful for assessing the expression level, gene copy or genome profile of a biomarker in Tables 1, 2, 3, 4 and/or 13 or gene alteration described herein.

The term “primer” as used herein refers to a nucleic acid sequence, whether occurring naturally as in a purified restriction digest or produced synthetically, which is capable of acting as a point of synthesis of when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art. The term “primer” as used herein refers a set of primers which can produce a double stranded nucleic acid product complementary to a portion of the RNA products of the biomarker or sequences complementary thereof.

The term “probe” as used herein refers to a nucleic acid sequence that will hybridize to a nucleic acid target sequence. In one example, the probe hybridizes to an RNA product of the biomarker or a nucleic acid sequence complementary thereof. The length of probe depends on the hybridize conditions and the sequences of the probe and nucleic acid target sequence. In one embodiment, the probe is at least 8, 10, 15, 20, 25, 50, 75, 100, 150, 200, 250, 400, 500 or more nucleotides in length.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)2, scFv, dsFv, ds-scFv, dimers, minibodies, diabodies, and multimers thereof and biospecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, biospecific antibody fragments and other fragments can also be synthesized by recombinant techniques.

The definitions and embodiments described in particular sections are intended to be applicable to other embodiments herein described for which they are suitable as would be understood by a person skilled in the art.

### II. METHODS

Disclosed herein are biomarkers, which are differentially expressed according to classification and/or prognosis in subjects with lung adenocarcinoma, including biomarkers whose gene copy number and/or expression level is increased, and biomarkers whose gene copy number and/or expression level is decreased. The biomarkers whose gene copy number and/or expression level is increased include, in one embodiment, one or more of the genes listed in Tables 1 and/or 3 and biomarkers whose gene copy number and/or expression level is decreased include in one embodiment, one or more of the genes listed in Table 2 and/or 4. Comparing biomarker gene copy number and/or expression level of one or more of these biomarkers to a control wherein the control optionally comprises a reference profile is useful for classifying a subject as belonging to a BAC group or an invasive ADC group, diagnosing a subject as having BAC or invasive ADC and/or is prognostic for poor survival or good survival. Combinations of these biomarkers are useful for prognosing, diagnosing and classifying subjects.

In a first aspect, the application provides a method of classifying or prognosing a subject with lung adenocarcinoma, comprising the steps:

(a) determining the expression level of a biomarker in a test sample from the subject, wherein each biomarker corresponds to a gene listed in Table 1, 2, 3 and/or 4,

(b) comparing the expression level of the one or more biomarkers with a control,

wherein a difference or a similarity in the expression level of the one or more biomarkers between the one or more controls and the test sample is used to classify the subject with lung adenocarcinoma into BAC or invasive ADC group and/or prognose the subject with lung adenocarcinoma into a poor survival group or a good survival group.

In another embodiment, the application provides methods for diagnosis. In an embodiment a method for diagnosing a subtype of lung adenocarcinoma in a subject is provided, the steps comprising:

(a) determining the expression level of one or more biomarkers in a test sample from the subject, wherein each biomarker corresponds to a gene in Table 1, 2, 3 and/or 4;

(b) comparing the expression of the one or more biomarkers with a control,

wherein a difference or a similarity in the expression of the one or more biomarkers between the test sample and the control is used to diagnose the subject has having BAC or invasive ADC.

The expression level of a biomarker can be determined for example by contacting the sample comprising nucleic acids (eg nucleic acid test sample) or polypeptides (e.g. polypeptide test sample) with a detection agent, such as a probe, primer set or antibody, to form for example a complex between the detection agent and the transcription product to thereby determine the level of expression of the biomarker (e.g. for comparison to control).

Another embodiment provides a method comprising;

(a) obtaining a nucleic acid test sample from a subject;

(b) contacting the sample with at least one nucleic acid probe to detect, or primer to amplify and identify the level of expression of one or more biomarkers selected from Tables 1, 2, 3, 4 and/or 13 in the subject's test sample,

wherein the level of expression of the one or more genes selected from Tables 1, 2, 3, 4 and/or 13 indicates the subtype of lung ADC, and/or classifies the subject as belonging to a BAC group or an invasive ADC group and/or indicates the subject has a poor prognosis or good prognosis.

Another embodiment provides a method comprising;

(a) obtaining a polypeptide test sample from a subject;

(b) contacting the sample with at least one antibody to detect the level of expression of one or more biomarkers selected from Tables 1, 2, 3, 4 and/or 13 in the subject's test sample,

wherein the level of expression of the one or more genes selected from Tables 1, 2, 3, 4 and/or 13 indicates the subtype of lung ADC, and/or classifies the subject as belonging to a BAC group or an invasive ADC group and/or indicates the subject has a poor prognosis or good prognosis.

In an embodiment, the one or more biomarkers correspond to one or more genes in Table 1 and/or 3, and wherein an increase in expression in one or more of the biomarkers is indicative the subject has invasive ADC.

In another embodiment, the one or more biomarkers correspond to one or more genes in Table 2 and/or 4 and wherein a decrease in expression of one or more biomarkers compared to the control is indicative the subject has invasive ADC.

In a further embodiment, the one or more biomarkers comprises one or more of the genes listed in Table 3, wherein an increase in the expression level of the one or more biomarkers compared to the control indicates the subject has invasive ADC.

In yet a further embodiment, the biomarkers comprise at least 2, 3, 4, 5, 6, 7, 8, 9, 10, 11, 12, 13, 14, 15, 16, 17, 18, 19, 20, 21, 22, 23, 24, 25, 26, or 27 or 28 of AP1S1, AP4M1, BRD9, CCDC21, CCL8, COPS6, CSDE1, EP300, GNB2, HIPK1, HRSP12, LAPTM4B, MCM7, MGC4677, OLFM2, POP7, PPA1, PDCD6, RABL4, RPL30, SERPINE1, SH3BGRL3, SLC25A17, ST13, TAF6, TLE3, TOB2, and ZNF561, wherein an increase in the expression level of the one or more biomarkers compared to the control indicates the subject has invasive ADC.

In another embodiment, the one or more biomarkers comprise SERPINE1, GNB2 and/or ST13 wherein an increase in the expression level of the one or more biomarkers compared to the control indicates the subject has invasive ADC.

In another embodiment, the one or more biomarkers comprises one or more of the genes listed in Table 4, wherein a decrease in the expression level of the one or more biomarkers compared to the control indicates the subject has invasive ADC.

In an embodiment, the subject with lung ADC is classified into a BAC or invasive ADC group or diagnosed as having BAC or invasive ADC. In another embodiment, BAC is non-invasive BAC. In another embodiment, the BAC is BAC with focal invasions. In another embodiment, the invasive ADC is AWBF.

In another embodiment, the subject with lung ADC is prognosed into a poor survival group or a good survival group.

The application discloses that the biomarkers are independently prognostic of outcome. These biomarkers are useful alone or in combination with other biomarkers disclosed herein. For example PDCD6 expression level has been found to be prognostic of poor survival.

Accordingly in one embodiment, the biomarker comprises PDCD6. In one embodiment, the subject PDCD6 expression level is increased significantly in a subject with poor survival compared to a control e.g., normal lung. In another embodiment, the subject PDCD6 expression level is increased significantly in a subject with poor survival compared to a control e.g., normal lung. In one embodiment, the significant difference is at least P<0.5%. In certain embodiments, the control comprises an average or mean expression level for more than one control, e.g., more than one normal lung or matched non-tumor sample. In one embodiment the increase is at least 25%, at least 50%, at least 75%, at least 100%, at least 2, at least 3, and/or 4 fold. In one embodiment the increase is at least 3 fold.

It was also determined that SERPINE1, GNB2 and/or ST13 expression is increased in subjects with poor outcome.

Accordingly, one embodiment of the application is a method of prognosing a subject with lung adenocarcinoma, comprising the steps:

(a) determining the expression of a biomarker in a test sample from the subject, wherein the biomarker comprises one or more of PDCD6, SERPINE1, GNB2, and ST13,

(b) comparing the expression of the one or more biomarkers a control,

wherein a difference or similarity in the expression of the one or more biomarkers between the control and the test sample is used to prognose the subject into a poor survival group or a good survival group.

In one embodiment, the biomarkers comprise at least 2 of PDCD6, SERPINE1, GNB2, and ST13.

In certain embodiments, the control is normal lung and/or non-tumor matched control and an increase in the expression of the one or more biomarkers between the test sample and the control is indicative that the subject with lung ADC is in a poor survival group. In other embodiments where the control is normal lung and/or non-tumor matched control, a similarity in the expression of the one or more biomarkers between the test sample and the control e.g. no or no statistical change, is indicative that the subject with lung ADC has a good survival.

In another aspect the application provides a method of classifying or prognosing a subject with lung adenocarcinoma, comprising the steps:
a) obtaining a subject biomarker expression profile in a sample of the subject;
b) obtaining one or more biomarker reference expression profiles associated with a disease subtype or prognosis, wherein the subject biomarker expression profile and the biomarker reference profile each has a plurality of values, each value representing the expression level of a biomarker, wherein each biomarker corresponds to a gene in Table 1, 2, 3 and/or 4; and
c) selecting the biomarker reference expression profile most similar to the subject biomarker expression profile, to thereby classify the subject as having BAC or invasive ADC or prognose the subject as having a poor survival or a good survival.

Another embodiment provides a method for diagnosing a subtype of lung adenocarcinoma in a subject comprising the steps:
a) obtaining a subject biomarker expression profile in a sample of the subject;
b) obtaining one or more biomarker reference expression profiles associated with a subtype of lung adenocarcinoma, wherein the subject biomarker expression profile and the biomarker reference profile each has a plurality of values, each value representing the expression level of a biomarker, wherein each biomarker corresponds to a gene in Table 1, 2, 3 and/or 4; and
c) selecting the biomarker reference expression profile most similar to the subject biomarker expression profile, to thereby diagnose the subject as having BAC or invasive ADC.

In another aspect, the methods are used for identifying patients with poor prognosis. The application demonstrates that various expression profiles are associated with poor survival. For example, increased expression of genes listed in Table 3 and decreased expression of genes listed in Table 4 are associated with poor survival.

Accordingly in one embodiment the application provides, a method of predicting poor prognosis in a subject with lung adenocarcinoma, comprising the steps:
a) obtaining a subject biomarker expression profile in a sample of the subject; and
b) comparing the subject biomarker expression profile to a control wherein the control is a biomarker reference expression profile associated with a poor prognosis, wherein the subject biomarker expression profile and the biomarker reference profile each has a plurality of values, each value representing the expression level of a biomarker, wherein each biomarker corresponds to a gene in Table 1, 2, 3 and/or 4;
   wherein similarity of the biomarker reference expression profile to the subject biomarker expression profile, predicts poor prognosis for the subject.

In one embodiment, the reference expression profile associated with poor survival comprises the expression level of at least one gene from Table 3. In one embodiment, the biomarker reference expression profile associated with poor survival comprises the expression level of 2 or more of AP1S1, AP4M1, BRD9, CCDC21, CCL8, COPS6, CSDE1, EP300, GNB2, HIPK1, HRSP12, LAPTM4B, MCM7, MGC4677, OLFM2, POP7, PPA1, PDCD6, RABL4, RPL30, SERPINE1, SH3BGRL3, SLC25A17, ST13, TAF6, TLE3, TOB2, or ZNF561.

In another embodiment, the reference expression profile associated with poor survival comprises the expression level of at least one gene from Table 4. In one embodiment, the biomarker reference expression profile associated with poor survival comprises the expression level of 2 or more of C5orf21, C5orf29, CACNA1D, CCDC13, CNTN6, CRTAP, DMXL1, EOMES, ERBB2IP, FEZF2, HRH1, LRAP, MEGF10, NPCDR1, PAM, PPWD1, RAB5A, SEMA6A, SFRS12, SNRK, TRIM36, TTC21A, ULK4, VIPR1, ZNF502.

The application further provides a method of predicting poor prognosis in a subject with lung adenocarcinoma, comprising the steps:

(a) determining the expression of one or more biomarkers in a test sample from the subject, wherein each biomarker corresponds to a gene listed in Table 1 and/or 3,

(b) comparing the expression of the one or more biomarkers to a control,

wherein an increase in expression of the one or more biomarkers between the test sample and the one or more controls is indicative of poor survival.

In one embodiment, the one or more biomarker is selected from Table 3. In one embodiment, the biomarkers comprise at least 2, 3, 4, 5, 6, 7, 8, 9, 10, 11, 12, 13, 14, 15, 16, 17, 18, 19, 20, 21, 22, 23, 24, 25, 26, 27, or 28 of the genes AP1S1, AP4M1, BRD9, CCDC21, CCL8, COPS6, CSDE1, EP300, GNB2, HIPK1, HRSP12, LAPTM4B, MCM7, MGC4677, OLFM2, POP7, PPA1, PDCD6 RABL4, RPL30, SERPINE1, SH3BGRL3, SLC25A17, ST13, TAF6, TLE3, TOB2, or ZNF561.

In another embodiment, the application provides a method of predicting poor prognosis in a subject with lung adenocarcinoma, comprising the steps:

(a) determining the expression level of one or more biomarkers in a test sample from the subject, wherein each biomarker corresponds to a gene listed in Table 2 or 4,

(b) comparing the expression level of the one or more biomarkers with a control,

wherein a decrease in expression of the one or more biomarkers between the test sample and the control is indicative of poor survival.

In another embodiment, the one or more biomarker is selected from Table 4. In one embodiment, the biomarkers comprise at least 2, 3, 4, 5, 6, 7, 8, 9, 10, 11, 12, 13, 14, 15, 16, 17, 18, 19, 20, 21, 22, 23, 24, or 25 of the genes C5orf21, C5orf29, CACNA1D, CCDC13, CNTN6, CRTAP, DMXL1, EOMES, ERBB21P, FEZF2, HRH1, LRAP, MEGF10, NPCDR1, PAM, PPWD1, RAB5A, SEMA6A, SFRS12, SNRK, TRIM36, TTC21A, ULK4, VIPR1, or ZNF502.

In certain embodiments, the biomarkers comprise at least 2 of the genes listed in Table 1, 2, 3, 4 and/or 13. In another embodiment, the biomarkers comprise at least 3, 4, 5, 6, 7, 8, 9, 10, 11, 12, 13, 14, 15, 16, 17, 18, 19, 20, 21, 22, 23, 24, 25 or more of the gene listed in Tables 1, 2, 3, 4 and/or 13. In a further embodiment, the biomarkers comprise at least 2, 3, 4, 5, 6, 7, 8, 9, 10, 11, 12, 13, 14, 15, 16, 17, 18, 19, 20, 21, 22, 23, 24, 25, 26 or 27 of the genes listed in Table 3. In a further embodiment, the biomarkers comprise at least 2, 3, 4, 5, 6, 7, 8, 9, 10, 11, 12, 13, 14, 15, 16, 17, 18, 19, 20, 21, 22, 23, 24, or 25 of the genes listed in Table 4. In another embodiment, the biomarkers comprise at least 2, 3-10, 11-20, 21-30, 31-40, 41-50, 51-60, 61-70, 71-80, 81-90, 91-100, 101-100, 111-113 of the genes listed in Table 1 or 2. In another embodiment, the biomarkers comprise more than 113 of the genes listed in Table 1.

In another aspect, the application relates to genomic alterations in subjects with pulmonary adenocarcinoma according to different disease subtypes and survival outcomes. These genomic alterations can be used to classify individuals into a BAC or invasive ADC group and/or prognose individuals with ADC into a poor survival group or a good survival group.

Accordingly, one aspect of the application is a method of classifying a subject with lung adenocarcinoma or diagnosing the subject with a subtype of lung ADC in a subject, comprising the steps:

(a) determining a genomic profile in a test sample from the subject,

(b) comparing the genomic profile to a control,

wherein a difference or a similarity in the genomic profile between the control and the test sample is used to classify the subject with lung ADC into a BAC group or an invasive ADC group or diagnose the subject as having BAC or invasive ADC.

In an embodiment, the genomic alteration and/or difference in the genomic profile is an amplification (e.g. increased gene copy compared to normal gene copy) in the test sample and is used to classify the subject with lung adenocarcinoma into non-invasive BAC with minimal risk to develop metastasis or die of the disease, or invasive ADC with risk to develop and die of recurrence and metastasis. In another embodiment, the genomic alteration and/or difference in the genomic profile is a deletion.

In another embodiment, the control comprises normal human tissue or cells, for example lung tissue or cells.

Another aspect provides a method of prognosing a subject with lung ADC, comprising the steps:

(a) determining a genomic profile in a test sample from the subject,

(b) comparing the genomic profile with a control,

wherein a difference or a similarity in the genomic profile between the control and the test sample is used to prognose the subject into a poor survival group or a good survival group.

In another embodiment, the application provides methods for diagnosis. In an embodiment a method for diagnosing a subtype of lung adenocarcinoma in a subject is provided, the steps comprising:

(a) determining the gene copy number of one or more biomarkers in a test sample from the subject, wherein each biomarker corresponds to a gene in Table 1, 2, 3 and/or 4;

(b) comparing the gene copy number of the one or more biomarkers with a control,

wherein a difference or a similarity in the gene copy number of the one or more biomarkers between the test sample and the control is used to diagnose the subject has having BAC or invasive ADC.

Another embodiment provides a method for diagnosing a subtype of lung adenocarcinoma in a subject comprising the steps:
a) obtaining a subject biomarker genomic profile in a sample of the subject;
b) obtaining one or more biomarker reference genomic profiles associated with a subtype of lung adenocarcinoma, wherein the subject biomarker genomic profile and the biomarker reference genomic profile each has a plurality of values, each value representing the expression level of a biomarker, wherein each biomarker corresponds to a gene in Table 1, 2, 3 and/or 4; and
c) selecting the biomarker reference genomic profile most similar to the subject biomarker expression profile, to thereby diagnose the subject as having BAC or invasive ADC.

Another embodiment provides a method comprising;

(a) obtaining a nucleic acid test sample from a subject;

(b) contacting the sample with at least one nucleic acid probe to detect, or primer to amplify and identify the level of expression or gene copy number of one or more biomarkers selected from Tables 1, 2, 3, 4 and/or 13 in the subject's test sample,

wherein the level of expression or gene copy number of the one or more genes selected from Tables 1, 2, 3, 4 and/or 13 indicates the subtype of lung ADC, and/or classifies the subject as belonging to a BAC group or an invasive ADC group and/or indicates the subject has a poor prognosis or good prognosis.

The gene copy number of a biomarker can be determined for example by contacting the sample comprising nucleic acids (eg nucleic acid test sample) with a detection agent, such as a probe, or primer set, to form for example a complex between the detection agent and the genomic region to thereby determine the gene copy number (or relative gene copy number) of the biomarker (e.g. for comparison to control). The control in one embodiment is a gene copy number fold change threshold.

In an embodiment, the control comprises a threshold level, for example a gene copy number fold change threshold, above which the subject is classified as belonging to an invasive ADC group, is diagnosed as having invasive ADC such as AWBF, and/or is prognosed as having poor survival. In an embodiment, the gene copy number fold change threshold is at least 1.9, at least 1.8, at least 1.7, at least 1.6, at least 1.5, at least 1.4, at least 1.3, at least 1.2, or at least 1.1. In an embodiment, the biomarker is selected from Table 12 and has a gene copy number fold change threshold of at 1.5.

Another embodiment provides a method comprising;

(a) determining the expression level, genomic alteration or gene copy number of one or more biomarkers in a test sample from the subject, wherein each biomarker corresponds to a gene in Table 1, 2, 3 and/or 4;

(b) normalizing the value of the expression level, genomic alteration or gene copy number to an internal control house keeping gene;

(c) comparing the normalized value for the expression level, genomic alteration or gene copy number of the one or more biomarkers with the average normalized expression value, genomic alteration or gene copy number of the one or more genes in a control,

(d) predicting the subtype of lung ADC, and/or the prognosis,

wherein a difference or a similarity in the normalized expression level, genomic alteration or gene copy number of the one or more biomarkers between the test sample and the control is used to diagnose the subject as having BAC or invasive ADC and/or to prognose the subject as having a poor prognosis or a good prognosis.

In an embodiment, the house keeping gene is selected from MAP2 (microtubule-associated protein 2) and B2M (beta-2-microglobulin), ACTB (Actin, beta), B2M, TBP (TATA box binding protein) and BAT1 (HLA-B associated transcript 1). The housekeeping gene can be used to normalize gene copy number and/or expression levels.

In an embodiment, the genomic alteration and/or difference in the genomic profile is an amplification (e.g. increased gene copy compared to normal gene copy) in the test sample and is used to prognose the subject with lung adenocarcinoma into a poor survival group or a good survival group. In another embodiment, the genomic alteration and/or difference in the genomic profile is a deletion.

In another embodiment, the control comprises normal human tissue or cells, for example lung tissue or cells.

The genome amplifications can comprise genes or portions thereof. For example amplified genes associated with increased tumor invasion and progression and/or higher gene content detected in subjects with AWBF compared with BAC include genes listed in Table 1, 3 and 13. Accordingly, in one embodiment, the genome amplification comprises one or more genes listed in Table 1, 3 or 13. In another embodiment, the genomic alteration comprises one or more of EPO, SERPINE1, SLC25A17, and POP7.

In a further embodiment, the genomic alterations comprise genome deletions. In one embodiment, the genome deletions comprise deletions in 3p, 5q, 4q and/or 6q. In a further embodiment, the genome deletions in 3p and 5q comprise one or more of the genes listed in Table 2.

As mentioned, the genomic alterations can comprise amplifications and deletions comprising genes or gene segments which result in gene copy number variations. For example, wherein a gene is amplified, the gain is referred to as a gene copy gain; wherein a gene is deleted, the deletion is referred to as a gene deletion. A subject without disease is typically diploid for genes in somatic cells. Accordingly the application provides a method of detecting gene copy number variations associated with lung ADC subtype and prognosis.

In one embodiment, the application provides a method of classifying a subject with lung adenocarcinoma and/or diagnosing the subject with a subtype of lung ADC, comprising the steps:
a) determining a gene copy number of one or more genes listed in Table 1 and/or 2 in a test sample of the subject;
b) comparing the gene copy number of each of the one or more genes in the test sample to one or more controls,
   wherein a difference in the gene copy number of the one or more the genes compared to control is used to classify the subject into a BAC group or an invasive ADC group and/or used to diagnose the subject as having BAC or invasive ADC. In an embodiment, an increase in gene copy number of a gene listed in Table 1 is indicative of belonging to an invasive ADC group. In another embodiment, a decrease in gene copy number of a gene listed in Table 2 is indicative of belonging to an invasive ADC group.

In another embodiment, the application provides a method of prognosing a subject with lung adenocarcinoma, comprising the steps:
c) determining a gene copy number of one or more genes listed in Table 1, 2, 3, 4 and/or 13 in a test sample of the subject;
d) comparing the gene copy number of each of the one or more genes in the test sample to one or more controls,
   wherein an increase in the gene copy number of the one or more genes of Tables 1, 3 and/or 13 or a decrease of the one or more genes of Tables 2 and/or 4 compared to control is indicative of poor prognosis.

In an embodiment, the one or more genes are selected from Tables 3, 4 and/or 13. In another embodiment, the genes are selected from EPO, SERPINE1, SLC25A17, and POP7.

In certain embodiment, the gene copy number of the control is a diploid gene copy number of the gene

A further aspect provides a method of predicting prognosis in a subject with lung adenocarcinoma, comprising the steps:
a. obtaining a subject gene copy number profile in a sample of the subject;
b. obtaining one or more reference gene copy number profiles associated with a prognosis, wherein the subject gene copy number profile and the reference gene copy number profile each has a plurality of values, each value representing the gene copy number of a gene in Table 1, 2, 3, 4 and/or 13; and
c. selecting the reference gene copy number profile most similar to the subject gene copy number profile, to thereby predict a prognosis for the subject.

In one embodiment, the genes are selected from Table 3 and/or 4. In another embodiment, the genes are selected from Table 13. In yet a further embodiment, the genes are selected from EPO, SERPINE1, SLC25A17, and POP7. In certain embodiments, the prognosis associated with the one or more reference gene copy number profiles comprise a poor survival group and a good survival group.

In yet a further embodiment, the application provides a method of classifying a subject with lung adenocarcinoma, comprising the steps:
a) determining a gene copy number profile in a test sample of a subject wherein the gene copy number profile comprises one or more gene copy gains of one or more genes listed in Table 1, 3 and/or 13 one or more gene copy deletions of one or more of the genes listed in Table 2 or 4;
b) comparing the gene copy number profile to one or more control reference profiles,
   wherein a difference or a similarity in the gene copy number profile between the test sample and the one or more controls is used to classify the subject with lung adenocarcinoma into a BAC group with minimal risk to develop metastasis or die of the disease, or invasive ADC group with risk to develop and die of recurrence and metastasis.

In another embodiment, the application provides a method of diagnosing a subtype of lung ADC in a subject with lung adenocarcinoma. The methods described herein are also useful for screening subjects for early diagnosis as described in the examples. In an embodiment, one or more biomarkers selected from the genes listed in Tables 1, 2, 3, 4 and 13 can be used with the methods described herein to screen a subject suspected of having lung cancer or lung ADC. In another embodiment, an expression profile or gene copy number profile of a subject suspected of having lung cancer or lung ADC is compared to a reference profile to determine if the subject has BAC or invasive ADC.

In certain embodiments, the one or more gene copy gains comprises TERT and/or PDCD6. In another embodiment, the one or more gene copy gains comprises 2 or more genes listed in Table 1, 3 and/or 13. In another embodiment, the one or more gene copy gains comprises at least 3, 4, 5, 6, 7, 8, 9 or 10 genes listed in Table 1, 3 and/or 13. In yet a further embodiment, the gene copy gains comprise gains in at least 10-20, or 20-30 genes listed in Table 1, 3 and/or 13. In another embodiment, the gene copy gains consist of gains in the genes listed in Table 3 or 13.

Another aspect provides detecting gene deletions. In one embodiment, the gene deletions comprise at least 2, 3, 4, 5, 6, 7, 8, 9 or 10 of the genes listed in Table 2 and/or 4. In another embodiment, the gene deletions comprise at least 10-20, or 20-30 of the genes listed in Table 2 and/or 4. In yet a further embodiment, the gene deletions comprise gains in at least 10-20, or 20-30 genes listed in Table 2. In another embodiment, the gene deletions consist of gains in the genes listed in Table 4.

In certain embodiments, the biomarkers or genes comprise at least 1 of the genes listed in Table 1, 2, 3, 4 and/or 13. In another embodiment, the biomarkers and/or genes comprise at least 2 of the genes listed in Table 1, 2, 3, 4 and/or 13. In another embodiment, the biomarkers comprise at least 3, 4, 5, 6, 7, 8, 9, 10, 11, 12, 13, 14, 15, 16, 17, 18, 19, 20, 21, 22, 23, 24, 25 or more of the gene listed in Tables 1, 2, 3, 4 or 13. In a further embodiment, the biomarkers comprise at least 2, 3, 4, 5, 6, 7, 8, 9, 10, 11, 12, 13, 14, 15, 16, 17, 18, 19, 20, 21, 22, 23, 24, 25, 26 or 27 of the genes listed in Table 3, and/or 13. In a further embodiment, the biomarkers comprise at least 2, 3, 4, 5, 6, 7, 8, 9, 10, 11, 12, 13, 14, 15, 16, 17, 18, 19, 20, 21, 22, 23, 24, or 25 of the genes listed in Table 4. In another embodiment, the biomarkers comprise at least 2, 3-10, 11-20, 21-30, 31-40, 41-50, 51-60, 61-70, 71-80, 81-90, 91-100, 101-100, 111-113 of the genes listed in Table 1 or 2. In another embodiment, the biomarkers comprise more than 113 of the genes listed in Table 1.

### III. LUNG CANCER STAGES AND TYPES

BAC and AWBF are subtypes of pulmonary adenocarcinoma which vary in disease outcome. BAC has good survival approaching 100% by certain assessments whereas AWBF has a relatively poor 5 year survival rate. It was demonstrated that BAC and AWBF have different genomic profiles. For example, it was shown that the 28 genes listed in Table 3 and the 25 genes listed in Table 4 are prognostic in ADC stage I patients. Further 33 genes listed in Table 13 have a higher gene content in AWBF compared with BAC and are therefore useful for diagnosing the subtype of lung ADC and prognosing survival, tumor invasion and progression. A ranking of these genes is provided in Table 13. The genes in Table 13 are ranked according to their diagnostic utility. For example, EPO, SERPINE1, SLC25A17, POP7 were found to have maximal ROC AUC and minimal QPCR copy number fold change threshold. Further it was demonstrated that PDCD6 is prognostic in ADC stages I-II and in the entire group stage I-II NSCLC. Accordingly, the application provides in certain embodiments methods that are useful for classifying, diagnosing, screening and/or identifying subjects with BAC. The application also demonstrates that the methods disclosed herein are useful for prognosing survival in early stage adenocarcinoma. Accordingly in one embodiment, the methods disclosed herein diagnose, prognose or classify a subject having or suspected of having lung adenocarcinoma in stage I-II lung adenocarcinoma.

In one embodiment, increased PDCD6 expression in stage I-II ADC is prognostic for poor survival.

In addition to the methods disclosed being prognostic in lung adenocarcinoma, they are also useful for predicting prognosis in subjects with non-small cell lung cancer (NSCLC). For example, PDCD6 is useful for prognosing subjects with NSCLC. Accordingly in one embodiment, the application provides a method of predicting poor prognosis in a subject with NSCLC comprising the steps:

(a) determining the expression level of PDCD6 in a test sample from the subject,

(b) comparing the expression level of PDCD6 with one or more controls, wherein an increase in expression of PDCD6 between the test sample and the one or more controls is indicative of poor survival.

In another embodiment, the PDCD6 gene copy number is assessed according to a method described herein wherein increased PDCD6 gene copy number is indicative of poor survival.

The methods described herein for classifying and diagnosing lung ADC subjects and prognosing survival can be combined with other methods for classifying, diagnosing and/or prognosing subjects with lung ADC such as other methods described herein or known in the art. A person skilled in the art would understand for example, that classification methods described herein can be combined with other methods of classifying and/or diagnosing lung ADC subtypes to obtain a confirmed and/or more accurate diagnosis. Similarly, other methods of prognosing survival can be combined with the methods described herein for more accurate prediction of survival.

### IV. SELECTING TREATMENT AND METHODS OF TREATMENT

In another aspect, the application provides a method of selecting a treatment for a subject with lung ADC.

Accordingly, the application provides a method of selecting a treatment for a subject with adenocarcinoma, comprising the steps:
a) prognosing the subject with lung adenocarcinoma into a poor survival group or a good survival group according to a method described herein wherein each group is associated with a treatment;
b) selecting the treatment associated with the group comprising the subject.

In an embodiment, the application provides a method of selecting a treatment for a subject with lung ADC, the method comprising:

(a) determining the expression level or gene copy number of one or more biomarkers in a test sample from the subject, wherein each biomarker corresponds to a gene in Table 3, 4 and/or 13;

(b) comparing the expression level or gene copy number of the one or more biomarkers with a control,

(c) selecting chemotherapy or surgery when the subject has an increase in the expression level of one or more biomarkers from Table 3, a decreased expression of one or more biomarkers from Table 4 and/or an increase in the copy number of one or more biomarkers from Table 13.

Another embodiment provides a method of selecting a treatment for a subject with lung ADC comprising the steps:
a) obtaining a subject biomarker expression or genomic profile in a sample of the subject;
b) obtaining one or more biomarker reference expression or genomic profiles associated with a subtype of lung adenocarcinoma, wherein the subject biomarker expression or genomic profile and the biomarker reference profile each has a plurality of values, each value representing the expression level or gene copy number of a biomarker, wherein each biomarker corresponds to a gene in Table 3, 4 and/or 13;
c) selecting the biomarker reference expression profile most similar to the subject biomarker expression profile; and
d) selecting chemotherapy or surgery when the subject has a biomarker expression profile that is similar to an aggressive ADC biomarker reference profile.

In another embodiment, the application provides a method of selecting a treatment for a subject with lung adenocarcinoma comprising the steps:
(a) determining the expression of a biomarker in a test sample from the subject, wherein the biomarker comprises one or more of the genes listed in Table 3, 4 and/or 13.
(b) comparing the expression of the one or more biomarkers with a control,
(c) prognosing the subject in a poor survival group or a good survival group wherein each group is associated with a treatment, wherein a difference or a similarity in the expression of the one or more biomarkers between the one or more controls and the test sample is used to classify the subject with lung adenocarcinoma into a poor survival group or a good survival group wherein each group is associated with a treatment,
(d) selecting the treatment associated with the group comprising the classified subject.

In an embodiment, the application provides a method of treating a subject with lung ADC, the method comprising:

(a) determining the expression level or gene copy number of one or more biomarkers in a test sample from the subject, wherein each biomarker corresponds to a gene in Table 3, 4 and/or 13;

(b) comparing the expression level and/or gene copy number of the one or more biomarkers with a control,

(c) treating the subject with chemotherapy or surgery when the subject has an increase in the expression level of one or more biomarkers from Table 3 a decreased expression of one or more biomarkers from Table 4, and/or an increase in the gene copy number of one or more biomarkers listed in Table 13.

Another embodiment provides a method of treating a subject with lung ADC comprising the steps:
a) obtaining a subject biomarker expression profile in a sample of the subject;
b) obtaining one or more biomarker reference expression profiles associated with a subtype of lung adenocarcinoma, wherein the subject biomarker expression profile and the biomarker reference profile each has a plurality of values, each value representing the expression level of a biomarker, wherein each biomarker corresponds to a gene in Table 3 and/or 4;
c) selecting the biomarker reference expression profile most similar to the subject biomarker expression profile; and
d) treating the subject with chemotherapy or surgery when the subject has a biomarker expression profile that is similar to an aggressive ADC biomarker reference profile.

Another embodiment provides use of chemotherapy or surgery to treat a subject with invasive ADC, comprising:

(a) determining the expression level or gene copy number of one or more biomarkers in a test sample from the subject, wherein each biomarker corresponds to a gene in Table 3, 4 and/or 13; and

(b) comparing the expression level or gene copy number of the one or more biomarkers with a control,

wherein chemotherapy or surgery is indicated when the subject has an increase in the expression level of one or more biomarkers from Table 3, a decrease in the expression level of one or more biomarkers from Table 4 and/or an increase gene copy number of one or more biomarkers listed in Table 13.

In another embodiment, the application provides use of chemotherapy or surgery to treat a subject with invasive ADC comprises:
a) obtaining a subject biomarker expression profile in a sample of the subject;
b) obtaining one or more biomarker reference expression profiles associated with a subtype of lung adenocarcinoma, wherein the subject biomarker expression profile and the biomarker reference profile each has a plurality of values, each value representing the expression level of a biomarker, wherein each biomarker corresponds to a gene in Table 3 and/or 4; and
c) selecting the biomarker reference expression profile most similar to the subject biomarker expression profile;
   wherein chemotherapy or surgery is indicated when the subject has a biomarker expression profile that is similar to an aggressive ADC biomarker reference profile.

For example treatments associated with good survival include local radiation and limited/localized surgery, localized treatment (radiofrequency ablation), whereas treatments associated with poor survival include surgery and/or chemotherapy and/or targeted therapy (biopathway targeting, drugs). In an embodiment the treatment selected for a subject identified as having BAC or in the good survival group comprises local radiation and limited/localized surgery, localized treatment (radiofrequency ablation). In another embodiment, the treatment selected for a subject identified as having aggressive ADC or in the poor survival group comprises surgery and/or chemotherapy and/or targeted therapy (biopathway targeting, drugs).

### V. Samples and Controls

The test sample and/or control can be any fluid, cell or tissue sample from a subject which can be assayed for biomarker expression products, particularly genes differentially expressed in subjects with ADC according to survival outcome and/or for which the genomic profile can be determined, including detecting genomic alterations, and gene copy number variations. In one embodiment, the test sample is a cell, cells or tissue from a tumor biopsy from the subject. In an embodiment, the test sample comprises a tissue sample comprising at least one tumor cell. For example, methods for detecting gene expression in a single cell are known in the art.

The sample and/or control is in one embodiment tumor tissue and/or cells, derived from, for example, lung biopsy, for example obtained by bronchoscopy, needle aspiration, thoracentesis and/or thoracotomy, and/or derived from cells found in sputum. In an embodiment, the sample and control are similar or the same sample type, eg both are lung biopsies.

The biomarker expression levels described herein can be determined by for example immunohistochemical staining and/or in situ. Accordingly in one embodiment, the test sample comprises a tissue sample suitable for immunohistochemistry or in situ hybridization.

The test sample and the control (e.g. reference profiles) can be similar sample types for example they can both comprise tumor cells from a subject with ADC. In another embodiment, the control can be an actual sample from a subject known to have ADC and good survival outcome or known to have ADC and have poor survival outcome. More specifically, in one embodiment, the control can be an actual sample from a subject known to have BAG, or known to have AWBF. The control is in certain embodiments, a normal or non-tumor cell sample. As mentioned previously, the control can be a threshold value, such as a gene copy number fold change threshold, and/or a previously determined expression or gene copy number.

A person skilled in the art will appreciate that the comparison between the genomic profile, gene copy number profile, and/or expression of the biomarkers in the test sample and the reference genomic profile, reference gene copy number profile, reference expression profile and/or expression level of the biomarkers in the control will depend on the control used. For example, if the control is from a subject known to have ADC and poor survival, and there is a difference in the genomic profile and/or expression of the biomarkers between the control and test sample, then the subject can be prognosed or classified in a good survival group. If the control is from a subject known to have ADC and good survival, and there is a difference in the genomic profile and/or expression of the biomarkers between the control and test sample, then the subject can be prognosed or classified in a poor survival group. For example, if the control is from a subject known to have ADC and good survival, and there is a similarity in the genomic profile and/or expression of the biomarkers between the control and test sample, then the subject can be prognosed or classified in a good survival group. For example, if the control is from a subject known to have ADC and poor survival, and there is a similarity in the genomic profile and/or expression of the biomarkers between the control and test sample, then the subject can be prognosed or classified in a poor survival group.

### VI. DETECTING METHODS

A person skilled in the art will appreciate that a number of methods can be used to detect or quantify the level of RNA products of the biomarkers within a sample, including microarrays, RT-PCR (including quantitative RT-PCR), nuclease protection assays and Northern blot analyses and in situ hybridization.

In addition, a person skilled in the art will appreciate that a number of methods can be used to determine the amount of a protein product of the biomarker of the application, including immunoassays such as Western blots, ELISA, and immunoprecipitation followed by SDS-PAGE and immunocytochemistry.

In addition, a person skilled in the art will appreciate that a number of methods can be used to detect or quantify genomic alterations and gene copy number variations such as amplifications and deletions including array comparative genome hybridization, quantitative PCR (qPCR) and FISH.

Accordingly in one embodiment, determining the biomarker expression level comprises use of RT-qPCR, expression array (for example the U122 plus 2 array) or immunohistochemistry In another embodiment, obtaining an expression profile comprises use of quantitative PCR (qPCR), RT-qPCR or an array. In certain embodiments, the array is a U122 plus 2 array.

In other embodiments, determining the biomarker expression level comprises use of an antibody.

In certain embodiments, the step of determining genome alteration or gene copy number comprises PCR and/or quantitative PCR (qPCR).

FISH analysis can also be utilized to detect genomic alterations. Accordingly in one embodiment, the step of determining the genome alteration or gene copy number comprises FISH analysis.

A person skilled in the art will appreciate that a number of detection agents can be used to determine the expression of the biomarkers. For example, to detect RNA products of the biomarkers, probes, primers, complementary nucleotide sequences or nucleotide sequences that hybridize to the RNA products can be used.

Similarly, a person skilled in the art will appreciate that a number of detection agents can be used to determine genomic alterations and gene copy number variations of the biomarkers. For example, to detect gene copy number, probes such as probes suitable for FISH, primers, complementary nucleotide sequences or nucleotide sequences that hybridize to the gene can be used.

To detect protein products of the biomarkers, ligands or antibodies that specifically bind to the protein products can be used.

Antibodies having specificity for a specific protein, such as the protein product of a biomarker, may be prepared by conventional methods. A mammal, (e.g. a mouse, hamster, or rabbit) can be immunized with an immunogenic form of the peptide, which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with the peptide and the monoclonal antibodies can be isolated.

A person skilled in the art will appreciate that the detection agents can be labeled.

The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

Conventional techniques of molecular biology, microbiology and recombinant DNA techniques, which are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Harnes & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995).

### VII. Compositions and Detection Agents

Another aspect of the application provides a composition for detecting a biomarker expression level or a genomic alteration.

Accordingly, one aspect provides a composition comprising a plurality of two or more isolated nucleic acid sequences, wherein each isolated nucleic acid sequence hybridizes to:
a) a biomarker RNA or DNA product of 2 or more gene listed in Tables 1, 2, 3, 4 and/or 13; and/or
b) a nucleic acid sequence complementary to a), and/or
c) a suitable carrier,
   wherein the composition is used to detect the level of RNA expression level of one or more genes or genomic alterations including gene copy number changes of one or more genes.

In one embodiment, the composition comprises a detection agent optionally an antibody, a probe or a primer, said detection agent binding a biomarker from Tables 1, 2, 3, and/or 4 and/or a suitable carrier.

In one embodiment the composition comprises primers that specifically amplify a gene or gene expression product listed in Tables 1, 2, 3, 4 and/or 13. In another embodiment, the composition comprises one or more probes that specifically bind to a gene, its expression product or the complement of either of a gene listed in Tables 1, 2, 3, 4 and/or 13. In one embodiment the composition comprises one or more primers listed in Table 10 and/or 11. In one embodiment, the composition comprises one or more primers listed in Table 10 for amplifying one or more of AP1S1, AP4M1, BRD9, CCDC21, CCL8, COPS6, CSDE1, EP300, GNB2, HIPK1, HRSP12, LAPTM4B, MCM7, MGC4677, OLFM2, POP7, PPA1, PDCD6, RABL4, RPL30, SERPINE1, SH3BGRL3, SLC25A17, ST13, TAF6, TLE3, TOB2, and/or ZNF561. In another embodiment, the composition comprises one or more primers listed in Table 11 for amplifying one or more of C5orf21, C5orf29, CACNA1D, CCDC13, CNTN6, CRTAP, DMXL1, EOMES, ERBB21P, FEZF2, HRH1, LRAP, MEGF10, NPCDR1, PAM, PPWD1, RAB5A, SEMA6A, SFRS12, SNRK, TRIM36, TTC21A, ULK4, VIPR1, and/or ZNF502. A person skilled in the art would readily be able to design additional primers that are suitable for quantitatively detecting gene alterations, gene copy number and biomarker expression level of one or more of the genes listed in Tables 1, 2, 3, 4 and/or 13.

In one embodiment, the composition comprises isolated nucleic acids which are useful for amplifying and/or hybridize to the RNA products of PDCD6, SERPINE1, GNB2 and/or ST13.

Another aspect provides a composition comprising a plurality of two or more detection agents such as antibodies, wherein each antibody specifically binds to a biomarker polypeptide product of 2 or more gene listed in Tables 1 and/or 2 wherein the composition is used to detect the level of biomarker polypeptide product of 2 or more genes.

### VIII. Arrays

Another aspect of the application provides an array for use in the methods described herein. In one embodiment, the application provides an array comprising a substrate having a plurality of addresses, wherein each address has disposed thereon a capture probe that can specifically bind a nucleic acid corresponding to each gene or a subset of genes listed in Tables 1, 2, 3, 4 and/or 13.

In another embodiment, the application provides an array comprising for each gene in a plurality of genes, the plurality of genes being at least 3 of the genes listed in Tables 1, 2, 3, 4 and/or 13 one or more polynucleotide probes complementary and hybridizable to an expression product in the gene. In one embodiment, the plurality of genes comprises the genes listed in Table 3 and/or 4. In another embodiment, the plurality of genes consists of the genes listed in Table 3, 4 and/or 13.

### IX. KITS

The application also provides for kits used to diagnose, prognose or classify a subject with ADC into a good survival group or a poor survival group that includes detection agents that can detect the expression products of the biomarkers.

In one embodiment, the application provides a kit to diagnose, prognose or classify a subject with ADC, comprising one or more detection agents that can detect the expression products of biomarkers, wherein the biomarkers comprise 1 or more of the genes in Tables 1, 2, 3, 4 and/or 13 and instructions for use.

Accordingly, the application includes a kit to diagnose, prognose or classify a subject with pulmonary adenocarcinoma, comprising detection agents that can detect the expression products of biomarkers, wherein the biomarkers comprise at least one biomarker listed in Tables 1, 2, 3, 4 and/or 13. In one embodiment, the biomarkers comprise at least one of PDCD6, SERPINE1, GNB2, and ST13.

The application also provides kits used to diagnose, prognose or classify a subject with ADC into a good survival group or a poor survival group that includes agents that can be used determine a genomic profile of a subject.

Accordingly, in one embodiment, the application provides a kit to diagnose, prognose or classify a subject with ADC, comprising one or more detection agents that can detect genomic alterations comprising genes, wherein the genes comprise 1 or more of the genes in Tables 1, 2, 3, 4 and/or 13 and instructions for use.

In another embodiment, the application provides a kit that can be used to diagnose, prognose or classify a subject with ADC into a good survival group or a poor survival group that includes agents that can be used to detect gene copy number variations.

In one embodiment, the application provides a kit to diagnose, prognose or classify a subject with early stage non-small cell lung cancer, comprising detection agents that can detect the expression products of biomarkers and instructions for use, wherein the biomarkers comprise one or more of PDCD6, SERPINE1, GNB2, and ST13.

In another embodiment, the application provides a kit to select a treatment for a subject with ADC, comprising one or more detection agents that can detect the expression products of biomarkers, wherein the biomarkers comprise one or more of the genes in Tables 1, 2, 3, 4 and/or 13 and instructions for use.

In an embodiment the kit comprises primers that specifically amplify a gene or gene expression product listed in Tables 1 and/or 2 and instructions for use. In another embodiment, the kit comprises one or more probes that specifically bind to a gene, its expression product or the complement of either of a gene listed in Tables 1 and/or 2 and instructions for use. In one embodiment the kit comprises one or more primers listed in Table 10 and/or 11 and instructions for use. In one embodiment, the kit comprises one or more primers listed in Table 10 for amplifying one or more of AP1S1, AP4M1, BRD9, CCDC21, CCL8, COPS6, CSDE1, EP300, GNB2, HIPK1, HRSP12, LAPTM4B, MCM7, MGC4677, OLFM2, POP7, PPA1, PDCD6, RABL4, RPL30, SERPINE1, SH3BGRL3, SLC25A17, ST13, TAF6, TLE3, TOB2, and/or ZNF561 and instructions for use. In another embodiment, the kit comprises one or more primers listed in Table 11 for amplifying one or more of C5orf21, C5orf29, CACNA1D, CCDC13, CNTN6, CRTAP, DMXL1, EOMES, ERBB21P, FEZF2, HRH1, LRAP, MEGF10, NPCDR1, PAM, PPWD1, RAB5A, SEMA6A, SFRS12, SNRK, TRIM36, TTC21A, ULK4, VIPR1, and/or ZNF502 and instructions for use.

The kit can also include a control or reference standard and/or instructions for use thereof. In addition, the kit can include ancillary agents such as vessels for storing or transporting the detection agents and/or buffers or stabilizers.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present application:

### EXAMPLES

### Example 1

Bronchioloalveolar carcinoma (BAC), a subtype of lung adenocarcinoma (ADC) without stromal, vascular or pleural invasion is considered an in situ tumor with 100% survival rate. However, the histological criteria for invasion remain controversial. BAC-like areas may accompany otherwise invasive adenocarcinoma, referred to as mixed type adenocarcinoma with BAC features (AWBF). AWBF are considered to evolve from BAC, representing a paradigm for malignant progression in ADC. However, the supporting molecular evidence remains forthcoming. The genomic changes of BAC and AWBF were studied by array comparative genomic hybridization (CGH). Using submegabase resolution tiling set array CGH, the genomic profiles of 14 BAC or BAC with focal area suspicious for invasion were compared to those of 15 AWBF. Threshold-filtering and Frequency-scoring analysis found that genomic profiles of non-invasive and focally-invasive BAC are indistinguishable, and show fewer aberrations than tumor cells in BAC-like area of AWBF. These aberrations occurred mainly at the sub-telomeric chromosomal regions. Increased genomic alterations were noted between BAC-like and invasive areas of AWBF. 113 genes that best differentiated BAC from AWBF were identified and were considered candidate marker genes for tumor invasion and progression. Correlative gene expression analyses demonstrated a high percentage of them as poor prognosis markers in early stage ADC. Quantitative polymerase chain reaction also validated the amplification and overexpression of PDCD6 and TERT on 5p, and the prognostic significance of PDCD6 in early stage ADC patients. Provided are novel candidate genes that may be responsible for and be markers for malignant progression in AWBF.

### Results

Most chromosomal changes in both BAC and AWBF were subtle indicating low levels of genomic alteration, as well as partial attenuation by contaminating non-neoplastic host cells. The profiles of BAC and BAC with focal areas suspicious for invasion were indistinguishable and showed low copy gains. AWBF had similar chromosomal changes but with greater variability and frequency and longer segmental alterations. Deletions were also more common in AWBF. In two patients with synchronous BAC and invasive AWBF, the BAC-like area of the latter showed greater aberrations than the BAC. In two other AWBF, greater alterations were also noted in invasive compared to BAC-like areas. Normal lung samples showed no alteration of these regions.

Using threshold-filtering, 119 clones that distinguished BAC from AWBF were identified. Hierarchical clustering of all cases using these clones separated BAC from AWBF samples In addition, a Fisher's Exact Test comparing the frequency of genomic changes between the BAC and AWBF groups yielded a list of 517 clones that best differentiated the two lesions. Integrating these two analyses was accomplished by applying a 10 clone “window” to identify shared regions. This resulted in a list of 256 candidate clones of high interest, from which a shorter list of 58 clones with gains in AWBF compared to BAC was selected. These clones included 113 unique amplified genes (Table 1) that could represent invasion and tumor progression markers for AWBF.

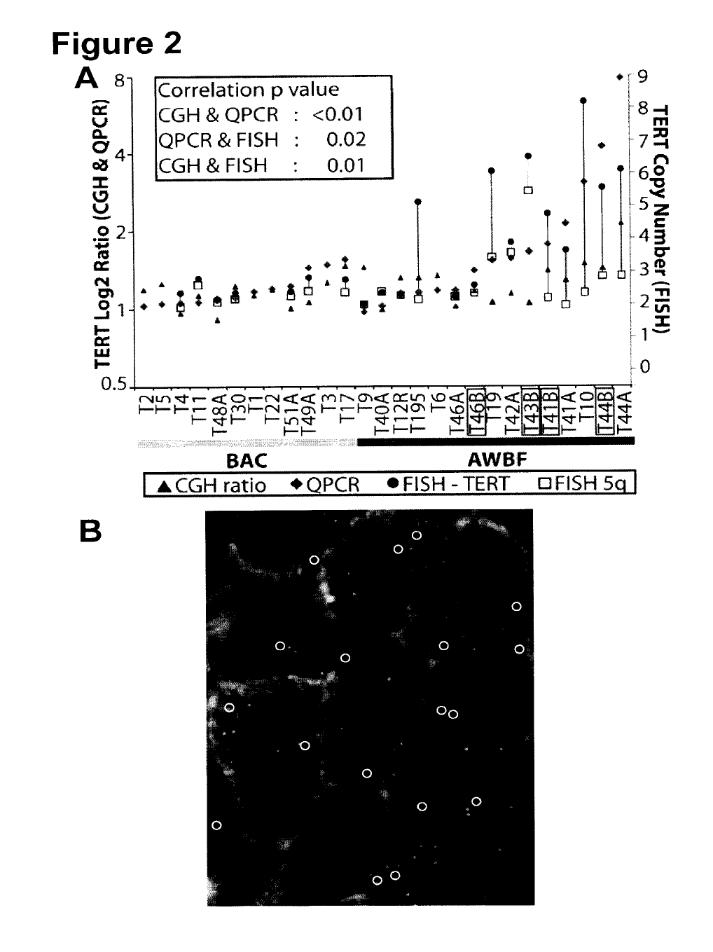

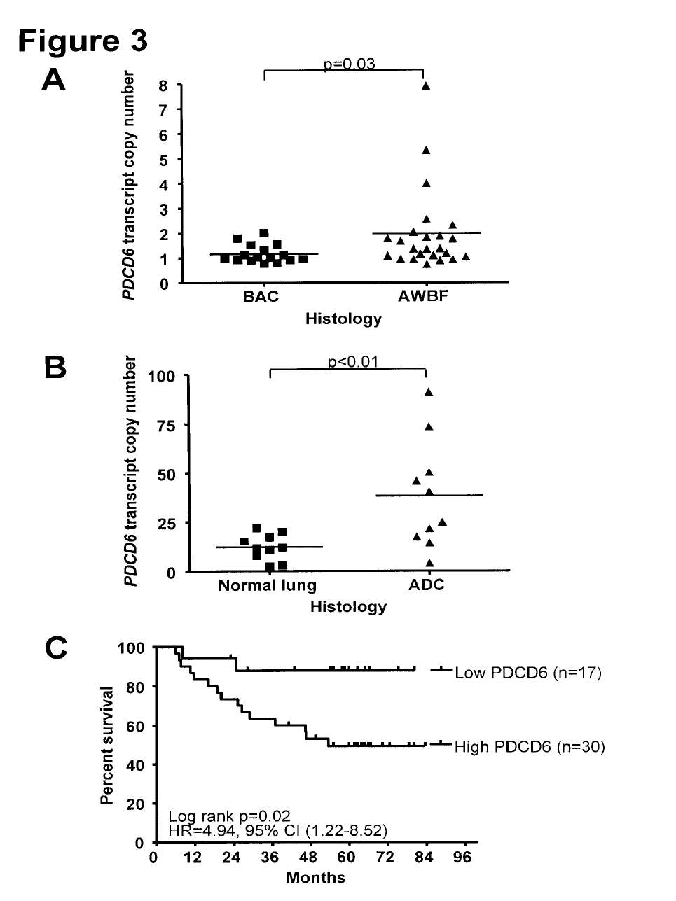

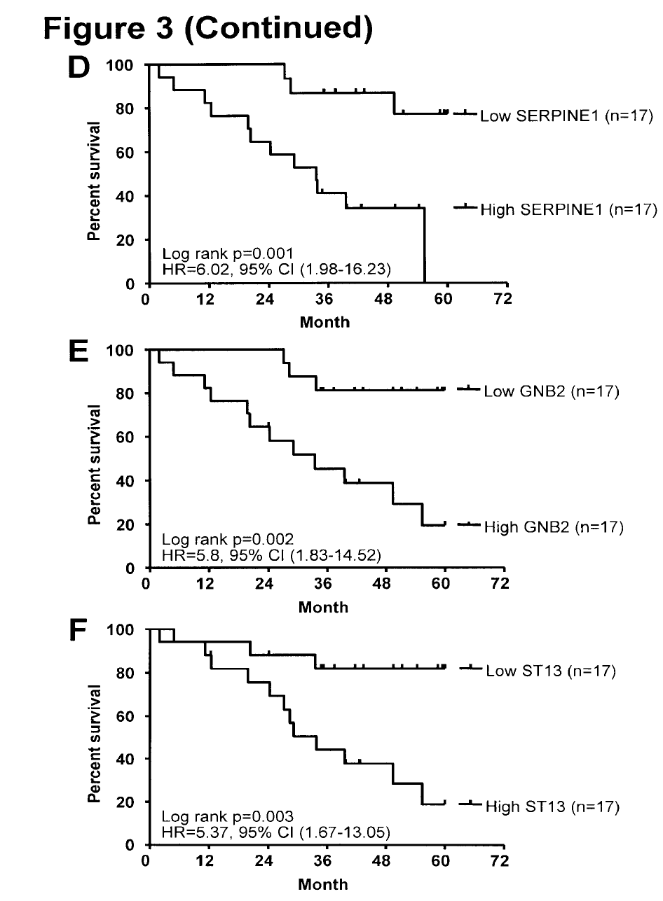

qPCR validated the gene content changes in 33 of the 113 candidate marker genes. Among the evaluated genes were TERT and PDCD6, which were selected for further validation by qPCR and/or FISH, based on their location on chromosome 5p that showed prominent genomic changes (FIG. 2B) Measurement of both genes by qPCR demonstrated significant differences in gene copy number between BAC and AWBF (p=0.03 for TERT and p=0.02 for PDCD6), consistent with the array CGH results (FIG. 3A). Using FISH, the gene copy of TERT and chromosome 5 was also studied in 21 tumors. The correlation coefficients were 0.76 between array CGH and qPCR, 0.50 between qPCR and FISH, and 0.53 between array CGH and FISH (FIG. 2). FISH appears the most sensitive in detecting the amplification levels and revealed the existence of chromosome 5 polysomy, especially in AWBF. Furthermore, FISH showed increased signal in the invasive area of AWBF compared to the BAC-like area in two samples (T41, T46, FIG. 2A). The coefficient of correlation for PDCD6 amplification between array CGH and qPCR was 0.94.

Using RT-qPCR, it was demonstrated that in 10 separate pairs of invasive ADC and their corresponding non-neoplastic lung tissues, PDCD6 was overexpressed in tumor compared to normal lung tissue (p<0.01), with a mean 3-fold increase in expression (FIG. 3B). In a series of 85 resected (stage I to IIIA) non-small cell lung carcinoma (NSCLC) samples, PDCD6 overexpression was an independent poor prognostic factor for overall survival in stage I-II ADC patients (HR=4.94, 95% CI 1.22-8.52, p=0.02) (FIG. 3C), as well as for the entire cohort of stage I-II NSCLC patients (HR=3.82, 95% CI 1.26-11.6, p=0.03).

A correlative gene expression study using external and internally available lung adenocarcinoma gene expression microarray datasets was then performed, starting with the 113 amplified genes. Analysis of the Toronto, Harvard and Michigan datasets discovered that 35%, 33% and 29% of the genes were overexpressed; a fraction are expected to be based on gene amplification. These datasets included only 87, 59 and 42 of the 113 genes, respectively, and overexpression was noted in 42%, 36% and 34% of them (Table 1 and Table 5). These results indicate a slight enrichment of the candidate amplified gene list for overexpression.

Univariate analysis of the Duke microarray dataset showed that 10,023 of 54,675 (18%) probe sets were prognostic for overall survival (p<0.05), with 4879 (9%) of overexpressed genes associated with poor prognosis. Among the 113 candidate amplified genes, 112 were represented by 227 probe sets on the U133 plus 2 array. The expression of 46/227 (20%) probe sets was significantly associated with prognosis. This is not significantly different from the overall proportion of all microarray probe sets that were prognostic (p=0.507). However, 34 of the 227 probe sets (15%), representing 27/113 (24%) putatively amplified and overexpressed genes (Table 3) were associated with poor prognosis. This is significantly higher than the 9% of all probe sets (p=0.002) with such association. The most prognostic overexpressed genes included SERPINE1 (HR=6.02, 95% Cl 1.98-16.23, p=0.001), GNB2 (HR=5.8, 95% CI 1.83-14.52, p=0.002) and ST13 (HR=5.37, 95% CI 1.67-13.05, p=0.003), (FIG. 3D-F).

Using frequency scoring the most common deletions were identified, as described below. The majority of deleted clones in AWBF were on 3p and 5q and they showed more continuity in their chromosomal location than on the other chromosomes. The deleted clones on chromosome 3p and 5q included 149 genes (Table 2), among which are FHIT and DLEC1. The 149 genes mapped to 441 probe sets on the U133 plus 2 array. The downregulation of the 28 probe sets (25 genes) (Table 4) was significantly associated with poor prognosis (HR<1). Similar to the candidate gained genes, correlative gene expression analysis using external and internally available lung adenocarcinoma datasets found that 22%, 20% and 16% of the genes in the Toronto, Harvard and Michigan datasets were downregulated. Among the 149 candidate genes with loss, only 113, 84 and 48 respectively were represented in these three datasets. Downregulation was found in 45%, 26% and 20% of them (Table 6). These results also showed an enrichment of the candidate deleted gene list for downregulation.

### Discussion

It was demonstrated that the genomic profile of BAC is distinguishable from that of invasive AWBF, with the latter displaying greater genomic aberrations. It was also demonstrated that there is progression at the genomic level from BAC-like to invasive areas of AWBF. The 113 differentially gained genes in AWBF compared to BAC represent candidate marker genes for tumor invasion and malignant progression. Correlative gene expression studies on microarray datasets suggest that a high percentage of these genes are prognostic markers for early stage ADC patients. Using qPCR, the common amplification of 25 genes including TERT and PDCD6 was validated, and PDCD6 overexpression was found to be an independent prognostic marker for poor overall survival in early stage ADC. Further validation may lead to use of these genes as markers for differentiating aggressive AWBF from non-invasive and prognostically excellent BAC.

The differential genomic changes noted between BAC and invasive AWBF provide important evidence for a better understanding of the pathogenesis of ADC. Two independent algorithms were used to enhance the certainty of the profile that distinguishes BAC from invasive AWBF. The inability to clearly differentiate BAC from BAC with focal area of invasion at the genomic level suggests that both may have a similar behavior with low metastatic potential, and early invasion is likely determined at gene expression levels by epigenetic mechanisms. The finding also suggests that BAC or BAC with focal invasion, which are negative for the overexpression of identified marker genes, could potentially be grouped into a single diagnostic entity with excellent prognosis (11, 15).

The 113 candidate marker genes that were identified may represent part of the “signature of chromosomal instability” for invasion and malignant progression in AWBF (22). The correlative gene expression validation rate (−35%) in the Harvard and Michigan datasets was limited by the low number of probesets in the microarray platform that matched the genomic gene list (less than half). Nevertheless, it confirms the importance of some of the candidate markers in lung carcinoma (Table 1) and the overexpression of others such as SAR1A (23), SYCP1 (24) and MCM7 (22) that have been linked to other malignancies as well as lung cancer. The poor prognostic significance of TERT gene amplification in NSCLC has been previously reported (25). The findings disclosed herein extend the importance of TERT amplification to AWBF and increased TERT gene copy due to chromosome 5 polysomy.

PDCD6, programmed cell death 6, or apoptosis-linked gene 2 (ALG-2) is located on chromosome 5pter-5p15.2 and is in close proximity to TERT. It encodes a 191 amino acid protein that was originally considered pro-apoptotic (26). PDCD6 belongs to the penta-EF hand Ca²⁺-binding protein family (27) and is ubiquitously expressed in the body. PDCD6 is required for T-cell receptor (TCR), glucocorticoid (26) and FAS (28) induced cell death. It interacts with the SH3-binding domain containing pro-apoptotic protein AIP1 (ALG-2-interacting protein-1) (29), peflin (30) and annexin XI (31) in a Ca²⁺-dependent way as well as with DAPK1 (death-associated protein kinase 1) (32). During FAS-induced apoptosis, PDCD6 which is a 22-kDa protein, is cleaved in its N-terminal to yield a 19-kD protein and translocates from the cytoplasmic membrane to the cytosol (28). More recent work questioned the need of PDCD6 for apoptosis, as it may be compensated by other functionally redundant proteins (33). Immunohistochemical staining has revealed high expression of PDCD6 in primary tumors compared to normal tissues of the breast, liver and lung (34, 35). Both nuclear and cytoplasmic over-expression have been reported for lung cancer, especially metastatic ADC, indicating that it plays a role in survival pathways (35). It has been demonstrated that PDCD6 is significantly overexpressed in lung ADC (35). Moreover, it has also been demonstrated herein that PDCD6 is a poor prognostic factor in both early stage NSCLC as well as ADC, and thus may serve as one of the markers to differentiate more indolent from aggressive AWBF.

Potti et al (19) reported a genomic strategy to refine prognosis for early stage NSCLC and identify patients at high risk of relapse after initial surgery. They constructed a lung metagene model based on gene expression data and showed that its prognostic accuracy surpasses that of a model based on traditional clinical data. Their model was applied to all histologic types of early stage disease but did not consider BAC as a special entity. Although none of the 122 genes in the published metagenes matched the 113 genes disclosed herein, analysis of the genes disclosed herein in their dataset showed that the overexpression of 27 genes (24%) was associated with poor prognosis in early stage ADC patients. Significantly higher gene copy number in AWBF compared with BAC was confirmed by qPCR on genomic DNA.

The 27 candidate markers that were identified include SERPINE1, GNB2, and ST13. SERPINE1, serpin peptidase inhibitor, clade E, member 1, also known as plasminogen activator inhibitor-1 (PAI1) is the primary physiological inhibitor of both tissue-type plasminogen activator (tPA) and urokinase-like PA (uPA), thus promotes the stabilization and formation of thrombi. Aside from regulating the fibrinolytic system, SERPINE1 has de-adhesive properties and is capable of inducing cell detachment that is dependent on the presence of complexes of uPA:uPA-receptor matrix-engaged integrins (36). Interestingly, SERPINE1 high expression has been linked previously with poor prognosis in a number of malignancies (37), including lung ADC (17). High expression of SERPINE1 may activate cellular scattering, promote migration and possibly enhance metastatic spread, all of which could account for the poor prognosis observed. The study relates the high expression to amplification present at the genomic level. SERPINE1 is located on the same locus, 7q21.3-q22, as GNB2, which is a novel prognostic marker for lung ADC. GNB2, guanine nucleotide-binding protein, beta-2, is the second of five possible genes encoding the beta-subunit of G proteins. As of yet, no other study associates GNB2 with lung cancer, but it is well established that G protein-coupled receptors can promote cancer progression and metastasis in a variety of tumors including NSCLC (38). ST13, suppression of tumorigenicity 13, whose aliases are P48, HOP and Hip (Hsc70-interacting protein), acts as a co-chaperone of heat-shock protein (Hsp) 70 to stabilizes its activity (39). Hsp70 is known to promote survival in cancer cells (40), thus making it is reasonable to hypothesize that ST13 amplification would lead to tumor progression. To date, ST13 has not been associated with NSCLC or its prognosis; hence it is another novel prognostic marker for lung ADC.

### Materials and Methods

### Tissue Sampling, DNA Isolation and Array CGH

Two 1 mm diameter cores were sampled for each tumor, and proper sampling was confirmed by post-coring HE section. DNA was isolated from tissue cores using standard phenol-chloroform method after Proteinase K (Roche, Laval, QC) digestion. The DNA was hybridized to the “27 K” high-density human bacterial artificial chromosome (hBAC) SMRT (Sub Megabase Resolution Tiling set) array CGH (BCCRC, Vancouver, BC), which contains two replicates of each hBAC clone. These arrays allow detection of 0.4 Mb single-copy gains and deletions even with 50% contamination of tumor by normal cells and up to 0.1 Mb in pure tumor samples (46). The hybridization, scanning and data processing were performed as previously described (42). Data was normalized with a three-step normalization framework (47) and log₂ ratio replicate data points that exceeded a standard deviation of 0.075 were excluded.

### Array CGH Data Analysis

Threshold Filtering:

The range of signal ratios recorded for normal samples defined the threshold by which a genuine genomic change was recognized and was calculated separately for each clone. A short list of 119 clones that best differentiate BAC from AWBF was created by filtering only clones that had array CGH ratio (aCGHR) above or below the threshold and with p value≦0.05 in Student's t-Test (two-tailed, unequal variance) followed by exclusion of any clone that had data for ≦4 tumors. Unsupervised hierarchical clustering using average linkage clustering of these selected clones by the Genesis software package (44) followed.

Frequency Scoring:

As previously described (42) array CGH data was smoothed by aCGH-Smooth (48) and the settings per chromosome of λ and breakpoints were 6.75 and 100, respectively. This data was displayed with Frequency Plot program, provided within the SeeGH software package. The frequency of gain loss and retention for each clone in the BAC group was compared to AWBF using the Fisher's Exact Test. A total of 517 clones with p value ≦0.05 were selected for further analysis.

Overlapping Threshold Filtering with Frequency Scoring:

To increase the certainty of clone selection and avoid exclusion of critical clones, the two short lists generated by Threshold filtering and Frequency scoring were overlapped. They were aligned according to their chromosomal location, any clone within 10 clone “window” created by at least one call from each of the short lists was selected and a list of 256 clones distributed in 34 continuous regions in 16 different chromosomes was obtained. Further manual selection based on log₂ ratio data of array CGH retrieved a shorter list of 58 clones that best differentiate BAC from AWBF. The analysis concentrated on clone gains rather than losses since they out numbered the latter. Annotations for genes of interest within these 58 clones were obtained from UCSC Genome Browser Gateway website (http://genome.ucsc.edu/cgi-bin/hgGateway) assembly April 2003 (49) via the SeeGH software (43). Relevant gene name and Entrez GeneID update was done based on information downloaded from Entrez Gene on Nov. 28, 2006 (http://www.ncbi.nlm.nih.gov/entrez/query.fcg i?CM D=search&DB=gene).

Deletions Analysis:

Based on Frequency scoring 8875 clones that were deleted in AWBF were identified. From them only 489 clones were deleted in at least a quarter of the AWBF cases. Annotations for the genes of interest within these 489 clones were obtained as described above for the gained clones. The sequences for each gene identified and referred to by Entrez Gene ID is incorporated herein by reference.

Validation by Realtime qPCR

Primer sets for genomic DNA were designed for exons of target genes and two housekeeping genes, MAP2 (microtubule-associated protein 2) and B2M (beta-2-microglobulin), chosen for their rare involvement in genomic alterations in lung cancer (Progenetix CGH database—http://www.progenetix.de/˜pgscripts/progenetix/Aboutprogenetix.html). Primer Express software v. 2.0 (Applied Biosystems, Foster City, Calif.) was used for the design of all primer sets. To exclude amplification of contaminating pseudogene sequences, primers (sequences provided in Table 10) were first aligned using the BLASTN program, followed by dissociation curve and primer efficiency tests. The qPCR assays were conducted in duplicate in 384-well plate using the SYBR Green assay in the ABI PRISM 7900-HT (Applied Biosystems, Foster City, Calif.) with 5 ng of genomic DNA (gDNA) in a 10 μl qPCR reaction. The reactions were activated at 95° C. for 10 minutes followed by 40 cycles of denaturing at 95° C. for 15s and annealing and extension at 60° C. for 1 min. The normalized, relative original copy number of each gene prior to the PCR procedure was calculated by the formula 2^{-ΔΔCt} (50) with the geometric mean of the two housekeeping genes serving as an endogenous reference, and the average of 8 normal lung samples as a calibrator.

Total RNA was isolated from fresh frozen tissues using the guanidium thiocyanate-phenol-chloroform method, DNAse I treated with DNA-free DNAse (Ambion, Austin, USA) and column purified using the RNeasy Mini Kit (Qiagen, Hilden, Germany). Five nanogram of total RNA were reverse transcribed using Superscript II Reverse Transcription reagents and Oligo dT (Invitrogen, Carlsbad, Calif.) to produce cDNA. The housekeeping genes ACTB (Actin, beta), B2M and TBP (TATA box binding protein) were used for the 10 paired samples and ACTB, B2M, TBP and BAT1 (HLA-B associated transcript 1) for the cohort of 94 tumors. PCR primer sets were designed as described above for qPCR on genomic DNA (sequences provided in the Table 10). Each of the realtime quantitative PCR amplifications were performed in a final volume of 10 μL in a 384-well plate, where a 5 ng equivalent of cDNA was used for the 10 paired samples and a 2 ng equivalent of cDNA was used for the 94 tumor cohort. All samples were run in duplicate. The reactions for the 10 paired samples were activated at 95° C. for 10 minutes followed by 40 cycles of denaturing at 95° C. for 15s and annealing and extension at 60° C. for 1 min. The reactions for the 94 tumor cohort were activated at 95° C. for 3 minutes followed by 40 cycles of denaturing at 95° C. for 15s, annealing at 65° C. for 15s and extension at 72° C. for 20s. Transcript number/ng cDNA was obtained using standard curves generated with a pool of 10 non-tumor lung genomic DNAs (51). Technical replicates were collapsed by averaging. Normalization and standardization of data was accomplished using the geometric mean of the expression levels of common house-keeping genes. The normalization method has been recently published (41).

### Validation by Fluorescent In Situ Hybridization (FISH)

FISH was performed using the TERT/5q dual-color FISH probe cocktail (Qbiogene, Montreal, QC) that contains the TERT locus (5p15) specific probe labelled directly with dGreen and the 5q31 (D5S89) specific probe directly labelled with Rhodamine, according to a published protocol (25). Fifty intact, non-overlapping tumor interphase nuclei were scored for TERT and 5q31 copy number. Results are presented as the mean gene copy number per nucleus.

### Correlative Gene Expression Validation

The ‘Harvard’ raw data was pre-processed with the RMA algorithm (52) in the R statistical environment (v2.1.1) using the affy package (v1.6.7) (53) of the BioConductor open-source library (54). Replicate arrays were collapsed by taking the arithmetic mean of their log₂ expression values. Pre-processed log₂ converted values for ADC were compared to normal lung values using SAM (v2.21) (55), and the number of positively- and negatively-regulated ProbeSets were determined with two-class unpaired analysis (median false detection rate (FDR)=3.98%).

The ‘Michigan’ raw data was pre-processed using RMAExpress (v0.3) (52, 56) followed by SAM (v2.21) analysis of tumor vs. normal (median FDR=4.98%).

The ‘Duke’ raw data was pre-processed using RMAExpress (v0.3) (52, 56) and log₂ transformed.

The ‘Toronto’ raw data from each chip was pre-processed separately using RMAExpress (v0.3) and log₂ converted (52, 56). The data for all samples was adjusted and merged using the Distance Weight Discrimination algorithm with ‘Standard output’ setting (57, 58). Duplicate data for the 4 tumor arrays which were profiled on both U133A and U133A2, were collapsed by taking the arithmetic mean of their adjusted expression values. The merged data was then used for SAM (v2.21) analysis of tumor vs. normal (median FDR=4.06%).

Genes from array CGH clones were matched to Affymetrix ProbeSets of all four studies based on LocusLink ID from array CGH data and Entrez GeneIDs from Affymetrix annotation Tables (Nov. 15, 2006. Release #21; https://www.affymetrix.com/analysis/releasedocs/netaffx_release 21.affx).

### Statistical Analysis

Pearson correlation coefficients for FISH were calculated using the ratio between mean TERT score and the mean control (5q) score.

The NSCLC cohort used for the mRNA expression study initially comprised of 94 samples. Nine cases that had equivalent survival curve as the remaining 85 cases had to be excluded from the study since they had no TBP or BAT1 read that was required for the normalization.

To study the association of PDCD6 mRNA expression with survival, overall survival (from date of surgery to date of death) of 85 NSCLC patients was used. PDCD6 adjusted expression was dichotomized at the 25^{th} percentile following identification of a distinctive survival pattern of this first quartile. Survival curves were plotted as the Kaplan-Meier graphs and compared using the log rank test. Univariate and multivariate analyses were done using the Cox proportional hazards regression model.

Survival analysis of 34 stage I ADC patients from the ‘Duke’ dataset was done using the Cox proportional hazards regression model. The genes whose expression was found to be significantly associated with prognosis were compared to the 113 candidate genes. The χ² test was used to compare the percentage of prognostic ProbSets and those predictive of poor prognosis between the entire microarray ProbSets and those corresponding to the 113 candidate genes. The expression of selected genes (SERPINE1, GNB2 and ST13) was dichotomized at the median in order to create Kaplan-Meier survival curves that were compared by the log rank test.

### Study Materials

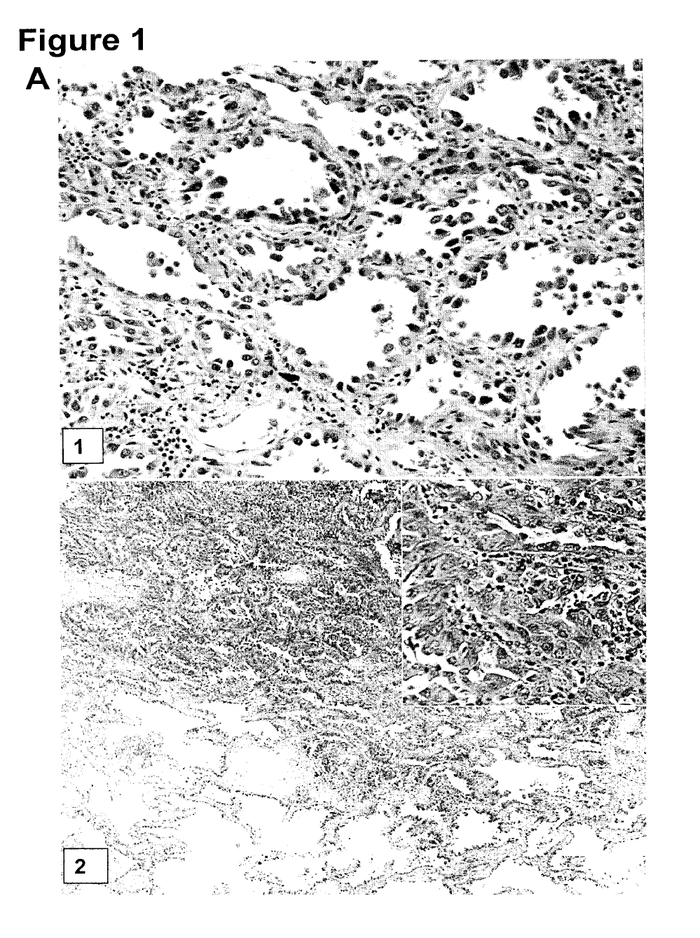

The study protocol was approved by the University Health Network Research Ethics Board and included 26 resected lung cancers (1996-2005) classified histologically as non-mucinous BAC or invasive-AWBF. For each case, the histology slides were reviewed independently by the study pathologists (SAR and MST) and tumors were classified according to the 2004 WHO criteria (2). Twelve cases were classified as AWBF when they had prominent non-mucinous BAC-like pattern (>50% of the tumor), but also had frank invasive adenocarcinoma of other histological types, such as acinar, papillary or solid (FIG. 1A). Fourteen cases were considered non-invasive BAC or BAC with possible focal micro-invasive area. In 11 of the AWBF cases, tissue from the BAC-like area was sampled, and in 3, additional tissue from a frankly invasive area was sampled separately. One case involved sampling from the invasive area only. Clinical characteristics of the samples are provided in Table 8. Eight corresponding normal lung tissues were selected arbitrarily as normal controls. For mRNA expression studies, matched tumor and normal tissues from the UHN snap-frozen lung tumor bank were used (41).

Tissue Sampling, DNA Isolation and Array CGH

DNA was isolated from formalin-fixed paraffin embedded (FFPE) tissue. Guided by Hematoxylin-eosin (HE) stained sections, representative paraffin blocks with tumor areas containing >50% tumor cell nuclei were marked and cored using the needle for tissue array (Beecher Instrument, Sun Prairie, Wis.). The process of tissue sampling, DNA isolation and array CGH is detailed below.

### Array CGH Data Analysis

Array CGH data analysis was based on two independent algorithms, Threshold-filtering and Frequency-scoring (42) using multiple software tools including SeeGH (43), Genesis (44), aCGH-Smooth (45) and Frequency-Plot (42). The algorithms and the overlap between them are described below. The analysis concentrated on clone gains rather than losses since clone gains involved more chromosomes, their prevalence was higher (FIG. 1B) and occasionally they were of higher copy number, not limited to just two copies per clone.

Validation by Realtime Quantitative PCR (qPCR)

Gene copy numbers were evaluated on DNA used in the array CGH studies by realtime qPCR using primer sets for target and house keeping genes. The evaluation of 33 genes including TERT and PDCD6 was performed on all the array CGH samples asides from two BACs (Table 12). The mRNA expression study was carried out on two groups of samples: 10 pairs of matched ADC and their adjacent normal lung tissue and 85 NSCLC samples. Primer sets design are included in Tables 10 and 11.

### Validation by Fluorescent In Situ Hybridization (FISH)

The 21 cases studied by FISH included 7 BAC with or without suspicion for invasion and 14 AWBF; three of the latter were scored in both their BAC and invasive areas. Additional case of AWBF was scored only in the invasive area. Among these cases is one with synchronous BAC and invasive AWBF sampled from the BAC area. FISH failed in 6 samples. The FISH protocol is detailed below.

### The Toronto DNA Microarray Dataset

RNA was extracted by phenol-chloroform method from 39 adenocarcinomas (Table 9) and 10 normal lung tissue samples. RNA quality was assessed by gel electrophoresis and Agilent Bioanalyzer. cRNA synthesis, hybridization and scanning were performed following the manufacturer's protocol. The adenocarcinomas RNA was profiled on Affymetrix U133A chip and the normal lung RNA on Affymetrix U133A2 chip. To ensure the compatibility of these 2 platforms, 4 of the 39 adenocarcinomas were re-profiled on the U133A2 chip.

### Correlative Gene Expression Study

The 113 amplified genes and the 149 deleted genes from array CGH analysis on the Toronto microarray dataset and on two publicly available lung cancer microarray expression datasets (17, 18) referred to as ‘Harvard’ and ‘Michigan’, respectively, were validated. For a detailed description of the analytic process and a summary of the validation see below, Tables 1 and 6.

In addition, univariate analysis was performed on microarray expression data of stage I ADC patient samples from a third dataset referred to as ‘Duke’ (19) in order to identify prognostic markers and compare them to the 113 candidate markers, as detailed in below and Table 7.

### Statistical Analysis

The Mann-Whitney test was used to compare the genomic copy number of 33 genes including TERT and PDCD6. Pearson correlation coefficients assessed the correlation between array CGH, qPCR and FISH results. The Wilcoxon signed rank test was used to compare PDCD6 expression in the paired ADC-normal samples. Survival analysis of PDCD6 mRNA of 85 NSCLC patients and 34 stage I ADC patients from the ‘Duke’ dataset is described above.

### Example 2

### Prognosing and Selecting Treatment

A heavy smoker patient joins a screening program for early diagnosis of lung cancer in high risk (heavy smokers) patients. A coin lesion for example, of 3.0 cm in the right upper lobe of lung is detected on chest CT scan. Right upper lobectomy is performed and a tumor with predominant bronchioloalveolar growth pattern is found. The tumor is associated with a large fibrotic area, where invasion is suspected. The differential diagnosis between BAC and AWBF is critical for the decision to administer adjuvant chemotherapy. At this point an additional section from the formalin-fixed paraffin embedded tumor block is cut and DNA is extracted. Quantitative PCR of the genomic DNA is run for 5 genes: PDCD6, TERT, SERPINE1, GNB2 and ST13.

The results are compared to control of normal lung tissue and show high content of PDCD6, SERPINE1 and GNB2 in the tumor. TERT is equivocally gained and ST13 shows normal content. Using additional section of the tumor FISH for TERT probe is performed and demonstrates clear amplification of TERT. Based on the ancillary studies, the tumor is diagnosed as AWBF with less favorable prognosis. Consequently the patient receives adjuvant chemotherapy.

### Example 3

Array CGH analysis of BAC and ADC identified one hundred and thirteen (113) genes as demonstrating differential frequencies of alteration in BAC and ADC. Thirty three (33) of these genes were further validated by Quantitative PCR analysis of gene copy number, and examined for potential diagnostic/prognostic utility (Table 13).

The Receiver Operating Characteristic (ROC) area under the curve (AUC) analysis was performed to determine the ability of each gene to separate the BAC and ADC samples into their appropriate diagnostic groups. Briefly, ROC analysis is based on comparison of true positive and false positive rates at various cut-offs. An ROC AUC value of 0.5 would indicate that the marker is no better than random chance at separating two groups, while a score of 1 would indicate that the marker is perfect at separating the two groups. Generally a marker with and AUC of 0.8 to 0.9 is considered good, while a AUC of 0.7 to 0.8 would represent a “fair” marker. Calculations were performed using the calculator at: http://www.rad.jhmi.edu/jeng/javarad/roc/JROCFITi.html (Eng J. ROC analysis: web-based calculator for ROC curves. Baltimore: Johns Hopkins University [updated 2006 May 17] Available from: http://www.jrocfit.org).

Although ROC analysis gives an indication of a marker's diagnostic value, it does not identify optimal cut-offs for maximal sensitivity and specificity. In order to generate relative risk and sensitivity/specificity scores for each gene, the QPCR copy number fold change threshold that gave maximal sensitivity while preserving a specificity of at least 90% was first identified. This was calculated on a per gene basis and a smaller threshold indicates both a lower copy number level and frequency of gains in BAC samples (for example a QPCR fold change threshold of 1.2 for PPA1 indicates that samples having greater than 1.2 copies of the gene (e.g a gain) are classified and diagnosed as having AWBF and/or prognosed as having poor survival with a 91.7% specificity and 53.3% sensitivity. Relative Risk is defined as the proportion of ADC samples with a gain divided by the proportion of BAC samples with a gain, as defined by the QPCR threshold identified above. This score thus represents the relative likelihood that a ADC will carry the alteration compared to a BAC. Similarly Sensitivity and Specificity are indicated for each gene.

Genes were prioritized based on a combination of maximal ROC value and minimal QPCR threshold. These genes represent the strongest diagnostic markers of ADC with minimal alterations in BAC patients (EPO, SERPINE1, SLC25A17, POP7).

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequence associated with each accession number provided herein is incorporated by reference in its entirely

### REFERENCES

1. Travis, W. D., Travis, L. B. & Devesa, S. S. (1995) Lung Cancer. Cancer 75: 191-202.
2. Travis, W. D., Brambilla, E. & Muller-Hermelink; H. K. (2004) in Lyon (IARC Press.
3. Kitamura, H., Karmeda, Y., Ito, T. & Hayashi, H. (1999) Atypical adenomatous hyperplasia of the lung. Implications for the pathogenesis of peripheral lung adenocarcinoma. Am J Clin Pathol 111: 610-22.
4. Kim, C. F., Jackson, E. L., Woolfenden, A. E., Lawrence, S., Babar, I., et al. (2005) Identification of bronchioalveolar stem cells in normal lung and lung cancer. Cell 121: 823-35.
5. Jackson, E. L., Willis, N., Mercer, K., Bronson, R. T., Crowley, D., et al. (2001) Analysis of lung tumor initiation and progression using conditional expression of oncogenic K-ras. Genes Dev 15: 3243-8.
6; Johnson, L., Mercer, K., Greenbaum, D., Bronson, R. T., Crowley, D., et al. (2001) Somatic activation of the K-ras oncogene causes early onset lung cancer in mice. Nature 410: 1111-6.
7. Guerra, C., Mijimolle, N., Dhawahir, A., Dubus, P., Barradas, M., et al. (2003) Tumor induction by an endogenous K-ras oncogene is highly dependent on cellular context. Cancer Cell 4: 111-20.
8. Miller, V. A., Kris, M. G., Shah, N., Patel, J., Azzoli, C., et al. (2004) Bronchioloalveolar pathologic subtype and smoking history, predict sensitivity to gefitinib in advanced non-small-cell lung cancer. J Clin Oncol 22: 1103-9,
9. Yokose, T., Suzuki, K., Nagai, K., Nishiwaki, Y., Sasaki, S., et al. (2000) Favorable and unfavorable morphological prognostic factors in peripheral adenocarcinoma of the lung 3 cm or less in diameter. Lung Cancer 29: 179-88.
10. Suzuki, K., Yokose, T., Yoshida, J., Nishimura, M., Takahashi, K., et al. (2000) Prognostic significance of the size of central fibrosis in peripheral adenocarcinoma of the lung. Ann Thorac Surg 69: 893-7.
11. Noguchi, M., Morikawa, A., Kawasaki, M., Matsuno, Y., Yamada, T., et al. (1995) Small adenocarcinoma of the lung. Histologic characteristics and prognosis. Cancer 75: 2844-52.
12. Rena, O., Papalia, E., Ruffini, E., Casadio, C., Filosso, P. L., et al. (2003) Stage I pure bronchioloalveolar carcinoma: recurrences, survival and comparison with adenocarcinoma of the lung. Eur J Cardiothorac Surg 23: 409-14.
13. Ebright, M. I., Zakowski, M. F., Martin, J., Venkatraman, E. S., Miller, V. A., et al. (2002) Clinical pattern and pathologic stage but not histologic features predict outcome for bronchioloalveolar carcinoma. Ann Thorac Surg 74: 1640-6; discussion 1646-7.
14. Breathnach, O. S., Kwiatkowski, D. J., Finkelstein, D. M., Godleski, J., Sugarbaker, D. J., et al. (2001) Bronchioloalveolar carcinoma of the lung: recurrences and survival in patients with stage I disease. J Thorac Cardiovasc Surg 121: 42-7.
15. Sakurai, H., Maeshima, A., Watanabe, S., Suzuki, K., Tsuchiya, R., et al. (2004) Grade of stromal invasion in small adenocarcinoma of the lung: histopathological minimal invasion and prognosis. Am J Surg Pathol 28: 198-206.
16. Petersen, I. & Petersen, S. (2001) Towards a genetic-based classification of human lung cancer. Anal Cell Pathol 22: 111-21.
17. Beer, D G., Kardia, S. L., Huang, C. C., Giordano, T. J., Levin, A. M., et al. (2002) Gene-expression profiles predict survival of patients with lung adenocarcinoma. Nat Med 8: 816-24.
18. Bhattacharjee, A., Richards, W. G., Staunton, J., Li, C., Monti, S., et al. (2001) Classification of human lung carcinomas by mRNA expression profiling reveals distinct adenocarcinoma subclasses. Proc Natl Acad Sci USA 98: 13790-5.
19. Potti, A., Mukherjee, S., Petersen, R., Dressman, H. K., Bild, A., et al. (2006) A genomic strategy to refine prognosis in early-stage non-small-cell lung cancer. N Engl J Med 355: 570-80.
20. Shibata, T., Uryu, S., Kokubu, A., Hosoda, F., Ohki, M., et al. (2005) Genetic classification of lung adenocarcinoma based on array-based comparative genomic hybridization analysis: its association with clinicopathologic features. Clin Cancer Res 11: 6177-85.
21. Murnane, J. P. & Sabatier, L. (2004) Chromosome rearrangements resulting from telomere dysfunction and their role in cancer. Bioessays 26: 1164-74.
22. Carter, S. L., Eklund, A. C., Kohane, I. S., Harris, L. N. & Szallasi, Z. (2006) A signature of chromosomal instability inferred from gene expression profiles predicts clinical outcome in multiple human cancers. Nat Genet 38: 1043-8.
23. Difilippantonio, S., Chen, Y., Pietas, A., Schluns, K., Pacyna-Gengelbach, M., et al. (2003) Gene expression profiles in human non-small and small-cell lung cancers. Eur J Cancer 39: 1936-47.
24. Tureci, O., Sahin, U., Zwick, C., Koslowski, M., Seitz, G., et al. (1998) Identification of a meiosis-specific protein as a member of the class of cancer/testis antigens. Proc Nat Acad Sci USA 95: 5211-6.
25. Zhu, C. Q., Cutz, J. C., Liu, N., Lau, D., Shepherd, F. A., et al. (2006) Amplification of telomerase (hTERT) gene is a poor prognostic marker in non-small-cell lung cancer. Br J Cancer 94: 1452-9.
26. Vito, P., Lacana, E. & D'Adamio, L (1996) Interfering with apoptosis: Ca(2+)-binding protein ALG-2 and Alzheimer's disease gene ALG-3. Science 271: 521-5.
27. Maki, M., Narayana, S. V. & Hitomi, K. (1997) A growing family of the Ca2+-binding proteins with five EF-hand motifs. Biochem J 328 (Pt 2): 718-20.
28. Jung, Y. S., Kim, K. S., Kim, K. D., Lim, J. S., Kim, J. W., et al. (2001) Apoptosis-linked gene 2 binds to the death domain of Fas and dissociates from Fas during Fas-mediated apoptosis in Jurkat cells, Biochem Biophys Res Commun 288: 420-6.
29. Vito, P., Pellegrini, L., Guiet, C. & D'Adamio, L. (1999) Cloning of AIP1, a novel protein that associates with the apoptosis-linked gene ALG-2 in a Ca2+-dependent reaction. J Biol Chem 274: 1533-40.
30. Kitaura, Y., Matsumoto, S., Satoh, H., Hitomi, K. & Maki, M. (2001) Peflin and ALG-2, members of the penta-EF-hand protein family, form a heterodimer that dissociates in a Ca2+-dependent manner. J Biol Chem 276: 14053-8.
31. Satoh, H., Shibata, H., Nakano, Y., Kitaura, Y. & Maki, M. (2002) ALG-2 interacts with the amino-terminal domain of annexin XI in a Ca(2+)-dependent manner. Biochem Biophys Res Commun 291: 1166-72.
32. Lee, J. H., Rho, S. B. & Chun, T. (2005) Programmed cell death 6 (PDCD6) protein interacts with death-associated protein kinase 1 (DAPk1): additive effect on apoptosis via caspase-3 dependent pathway. Biotechnol Lett 27: 1011-5.
33. Jang, I. K., Hu, R., Lacana, E., D'Adamio, L. & Gu, H. (2002) Apoptosis-linked gene 2-deficient mice exhibit normal T-cell development and function. Mol Cell Biol 22: 4094-100.
34. Krebs, J., Saremaslani, P. & Caduff, R. (2002) ALG-2: a Ca2+-binding modulator protein involved in cell proliferation and in cell death. Biochim Biophys Acta 1600: 68-73.
35. La Cour, J. M., Mollerup, J., Winding, P., Tarabykina, S., Sehested, M., et al. (2003) Up-regulation of ALG-2 in hepatomas and lung cancer tissue. Am J Pathol 163: 81-9.
36. Czekay, R: P. & Loskutoff, D. J. (2004) Unexpected role of plasminogen activator inhibitor I in cell adhesion and detachment. Exp Biol Med (Maywood) 229: 1090-6.
37. Andreasen, P. A., Kjoller, L., Christensen, L. & Duffy, M. J. (1997) The urokinase-type plasminogen activator system in cancer metastasis: a review. Int J Cancer 72: 1-22.
38. Dorsam, R. T. & Gutkind, J. S. (2007) G-protein-coupled receptors and cancer. Nat Rev Cancer 7: 79-94.
39. Nollen, E. A., Kabakov, A. E., Brunsting; J. F., Kanon, B., Hohfeld, J., et al. (2001) Modulation of in vivo HSP70 chaperone activity by Hip and Bag-1. J Biol Chem 276: 4677-82.
40. Ravagnan, L., Gurbuxani, S., Susin, S. A., Maisse, C., Daugas, E., et al. (2001) Heat-shock protein 70 antagonizes apoptosis-inducing factor. Nat Cell Biol 3: 839-43.
41. Barsyte-Lovejoy, D., Lau, S. K., Boutros, P. C., Khosravi, F., Jurisica, I., et al. (2006) The c-Myc oncogene directly induces the H19 noncoding RNA by allele-specific binding to potentiate tumorigenesis. Cancer Res 66: 5330-7.
42. Coe, B. P., Lee, E. H., Chi, B., Girard, L., Minna, J. D., et al. (2006) Gain of a region on 7p22.3, containing MAD1L1, is the most frequent event in small-cell lung cancer cell lines. Genes Chromosomes Cancer 45: 11-9.
43. Chi, B., DeLeeuw, R. J., Coe, B. P., MacAulay, C. & Lam, W. L. (2004) See GH—a software tool for visualization of whole genome array comparative genomic hybridization data. BMC Bioinformatics 5: 13.
44. Sturn, A., Quackenbush, J & Trajanoski, Z. (2002) Genesis: cluster analysis of microarray data. Bioinformatics 18: 207-8.
45. Jong, K., Marchiori E., Meijer, G., Vaart, A. V. & Ylstra, B. (2004) Breakpoint identification and smoothing of array comparative genomic hybridization data. Bioinformatics 20: 3636-7.
46. Garnis, C., Coe, B. P., Lam, S. L., MacAulay, C & Lam, W. L. (2005) High-resolution array CGH increases heterogeneity tolerance in the analysis of clinical samples. Genomics 85: 790-3.
47. Khojasteh, M., Lam, W. L., Ward, R. K. & MacAulay, C. (2005) A stepwise framework for the normalization of array CGH data. BMC Bioinformatics 6: 274.
48: Jong, K., Marohiori, E., Meijer, G., Vaart, A. V. & Ylstra, B. (2004) Breakpoint identification and smoothing of array comparative genomic hybridization data. Bioinformatics 20: 3636-7.
49. Karolchik, D., Baertsch, R., Diekhans, M., Furey, T. S., Hinrichs, A., et al. (2003) The UCSC Genome Browser Database. Nucleic Acids Res 31: 51-4.
50. Livak, K. J. & Schmittgen, T. D. (2001) Analysis of relative gene expression data using real-time quantitative PCR and the 2(-Delta Delta C(T)) Method. Methods 25: 402-8.
51. Yun, J. J., Heisler, L. E., Hwang, II, Wilkins, O., Lau, S. K., et al. (2006) Genomic DNA functions as a universal external standard in quantitative real-time PCR. Nucleic Acids Res 34: e85.
52. Irizarry, R. A., Bolstad, B. M., Collin, F., Cope, L. M., Hobbs, B., et al. (2003) Summaries of Affymetrix GeneChip probe level data. Nucleic Acids Res 31: e15.
53. Gautier, L., Cope, L., Bolstad, B. M. & Irizarry, R. A. (2004) affy—analysis of Affymetrix GeneChip data at the probe level. Bioinformatics 20: 307-15.
54. Gentleman, R. C., Carey, V. J., Bates, D. M., Bolstad, B., Dettling, M., et al. (2004) Bioconductor: open software developmedevet for computational biology and bioinformatics. Genome Biol 5: R80.
55. Tusher, V. G., Tibshirani, R. & Chu, G. (2001) Significance analysis of microarrays applied to the ionizing radiation response. Proc Natl Acad Sci U S A 98: 5116-21.
56. Bolstad, B. M., Irizarry, R. A., Astrand, M. & Speed, T. P. (2003) A comparison of normalization methods for high density oligonucleotide array data based on variance and bias. Bioinformatics 19: 185-93.
57. Benito, M, Parker, J, Du, Q, Wu, J, Xiang, D, Perou, CM & Marron, J S (2004) Adjustment of systematic microarray data biases. Bioinformatics 20:105-14.
58. Lu, Y, Lemon, W, Liu, P Y, Yi, Y, Morrison, C, Yang, P, Sun, Z, Szoke, J, Gerald, W L Watson, M, et al (2006) A gene expression signature predicts survival of patients with stage I non-small cell lung cancer. PLoS Med 3:e467.
59. Aviel-Ronen S, Coe B P, Lau S K, da Cunha Santos G, Zhu C Q, Strumpf D, Jurisica I, Lam W L, Tsao M S. (2008) Genomic markers for malignant progression in pulmonary adenocarcinoma with bronchioloalveolar features. Proc Natl Acad Sci USA. 2008 Jul. 22; 105(29):10155-60.

TABLE 1
The 113 amplified candidate marker genes
for tumor invasion and progression

| | | | | Up in | Up in |
|---|---|---|---|---|---|
| | Genomic | | Entrez | Harvard | Michigan |
| No. | location | Gene | Gene ID | data | data |
| 1 | 1p36.11 | CCDC21 | 64793 | | |
| 2 | 1p35-p34.3 | SH3BGRL3 | 83442 | | |
| 3 | 1p36.11 | UBXD5 | 91544 | | |
| 4 | 1p36 | CD52 | 1043 | | |
| 5 | 1p36.11 | AIM1L | 55057 | | |
| 6 | 1p36.11 | ZNF683 | 257101 | | |
| 7 | 1p36.11 | LIN28 | 79727 | | |
| 8 | 1p13.2 | AP4B1 | 10717 | | |
| 9 | 1p13.2 | DCLRE1B | 64858 | | |
| 10 | 1p13.2 | HIPK1 | 204851 | | |
| 11 | 1p13.2 | OLFML3 | 56944 | | |
| 12 | 1p22 | CSDE1 | 7812 | | |
| 13 | 1p13.2 | SIKE | 80143 | + | |
| 14 | 1p13-p12 | SYCP1 | 6847 | | |
| 15 | 2p11.2 | MGC4677 | 112597 | | |
| 16 | 2p11-q11 | PLGLB2 | 5342 | | + |
| 17 | 5pter-p15.2 | PDCD6 | 10016 | + | |
| 18 | 5p15.3 | AHRR | 57491 | | |
| 19 | 5p15.33 | EXOC3 | 11336 | + | |
| 20 | 5p15.3 | SLC9A3 | 6550 | + | + |
| 21 | 5p15.33 | CEP72 | 55722 | | |
| 22 | 5p15.3 | TPPP | 11076 | | |
| 23 | 5p15.33 | ZDHHC11 | 79844 | | |
| 24 | 5p15.33 | BRD9 | 65980 | | |
| 25 | 5p15.33 | TRIP13 | 9319 | + | + |
| 26 | 5p15.3 | NKD2 | 85409 | | |
| 27 | 5p15 | SLC12A7 | 10723 | | |
| 28 | 5p15.33 | TERT | 7015 | | + |
| 29 | 5pter-p15.3 | CLPTM1L | 81037 | | |
| 30 | 5p15.3 | SLC6A3 | 6531 | | + |
| 31 | 7q22.1 | ZNF3 | 7551 | | + |
| 32 | 7q22.1 | COPS6 | 10980 | + | |
| 33 | 7q21.3-q22.1 | MCM7 | 4176 | + | + |
| 34 | 7q22.1 | AP4M1 | 9179 | | |
| 35 | 7q22.1 | TAF6 | 6878 | + | + |
| 36 | 7q22.1 | MGC40499 | 245812 | | |
| 37 | 7q22 | FBXO24 | 26261 | | |
| 38 | 7q22 | PCOLCE | 5118 | | |
| 39 | 7q22 | MOSPD3 | 64598 | | |
| 40 | 7q22 | TFR2 | 7036 | + | |
| 41 | 7q22 | ACTL6B | 51412 | + | |
| 42 | 7q21.3- | GNB2 | 2783 | | + |
| | q22.1\|7q22 | | | | |
| 43 | 7q22 | PERQ1 | 64599 | | |
| 44 | 7q22 | POP7 | 10248 | + | |
| 45 | 7q22 | EPO | 2056 | + | + |
| 46 | 7q22 | ZAN | 7455 | | |
| 47 | 7q21.3-q22 | SERPINE1 | 5054 | | + |
| 48 | 7q22.1 | AP1S1 | 1174 | + | |
| 49 | 8q22.1 | LAPTM4B | 55353 | + | |
| 50 | 8q22 | MATN2 | 4147 | | |
| 51 | 8q22 | RPL30 | 6156 | + | |
| 52 | 8q22.2 | C8orf47 | 203111 | | |
| 53 | 8q22 | HRSP12 | 10247 | + | |
| 54 | 8q22.1 | POP1 | 10940 | | |
| 55 | 8q22.2 | NPAL2 | 79815 | | |
| 56 | 10p12.1 | MKX | 283078 | | |
| 57 | 10q22 | H2AFY2 | 55506 | | |
| 58 | 10q22.1 | AIFM2 | 84883 | | |
| 59 | 10q22.1 | TYSND1 | 219743 | | |
| 60 | 10q22.1 | SAR1A | 56681 | + | |
| 61 | 10q11.1-q24 | PPA1 | 5464 | | |
| 62 | 10q21-q22 | NPFFR1 | 64106 | | |
| 63 | 15q22 | TLE3 | 7090 | + | + |
| 64 | 17q11.2-q12 | CCL2 | 6347 | | |
| 65 | 17q11.2-q12 | CCL7 | 6354 | | |
| 66 | 17q21.1-q21.2 | CCL11 | 6356 | | |
| 67 | 17q11.2 | CCL8 | 6355 | | |
| 68 | 19p13.2 | ZNF561 | 93134 | | |
| 69 | 19p13.2 | ZNF562 | 54811 | | |
| 70 | 19p13.2 | FBXL12 | 54850 | | |
| 71 | 19p13.3 | UBL5 | 59286 | | |
| 72 | 19p13 | PIN1 | 5300 | | |
| 73 | 19p13.2 | OLFM2 | 93145 | | |
| 74 | 19q13.41 | ZNF702 | 79986 | | |
| 75 | 19q13.41 | ZNF347 | 84671 | | |
| 76 | 19q13.41 | ZNF665 | 79788 | | |
| 77 | 19q13.41 | VN1R2 | 317701 | | |
| 78 | 19q13.41 | VN1R4 | 317703 | | |
| 79 | 19q13.3-q13.4 | BIRC8 | 112401 | | |
| 80 | 20q11.2 | GHRH | 2691 | | + |
| 81 | 20q11.23-q12 | MANBAL | 63905 | | |
| 82 | 20q12-q13 | SRC | 6714 | + | + |
| 83 | 20q11.2-q12 | BLCAP | 10904 | | |
| 84 | 20q11.2-q12 | NNAT | 4826 | | |
| 85 | 22q13.1 | CACNG2 | 10369 | | |
| 86 | 22q13.1 | RABL4 | 11020 | + | |
| 87 | 22q12- | PVALB | 5816 | | |
| | q13.1\|22q13.1 | | | | |
| 88 | 22q13.1 | NCF4 | 4689 | | |
| 89 | 22q13.1 | CSF2RB | 1439 | | |
| 90 | 22q13.1 | TST | 7263 | | |
| 91 | 22q13.1 | MPST | 4357 | | |
| 92 | 22q12.3 | KCTD17 | 79734 | | |
| 93 | 22q12.3 | TMPRSS6 | 164656 | | |
| 94 | 22q13\|22q13.1 | IL2RB | 3560 | | |
| 95 | 22q13.2 | SLC25A17 | 10478 | + | |
| 96 | 22q13.2 | ST13 | 6767 | | |
| 97 | 22q13.2 | RBX1 | 9978 | | |
| 98 | 22q13.2 | EP300 | 2033 | | |
| 99 | 22q13\|22q13.2 | TEF | 7008 | + | + |
| 100 | 22q13.2-q13.31 | TOB2 | 10766 | | |
| 101 | 22q13.2 | PHF5A | 84844 | | |
| 102 | 22q11.2-q13.31\| | ACO2 | 50 | | |
| | 22q13.2-q13.31 | | | | |
| 103 | 22q13.2 | POLR3H | 171568 | | |
| 104 | 22q13.2-q13.31 | CSDC2 | 27254 | | |
| 105 | 22q13.2 | PMM1 | 5372 | | |
| 106 | 22q13 | SREBF2 | 6721 | | |
| 107 | 22q13.1-q13.31 | TNFRSF13C | 115650 | | |
| 108 | 22q13.2 | CENPM | 79019 | | |
| 109 | 22q13.2 | SEPT3 | 55964 | | |
| 110 | 22q13.2 | WBP2NL | 164684 | | |
| 111 | 22q13-qter\|22q11 | NAGA | 4668 | | |
| 112 | 22q13.2 | C22orf32 | 91689 | | |
| 113 | 22q13.2-q13.31 | NDUFA6 | 4700 | | |

TABLE 2
The 149 deletion candidate marker genes on 3p and 5q for A WBF

| No. | Genomic location | Gene | Entrez Gene ID |
|---|---|---|---|
| 1 | 3q29 | PPP1R2 | 5504 |
| 2 | 3q26.2-qter | APOD | 347 |
| 3 | 3p26-p25 | CNTN6 | 27255 |
| 4 | 3p26.3 | CRBN | 51185 |
| 5 | 3p26.2 | ARL8B | 55207 |
| 6 | 3p26.2 | EDEM1 | 9695 |
| 7 | 3p26.1-p25.1 | GRM7 | 2917 |
| 8 | 3p25.3-p25.2 | ATG7 | 10533 |
| 9 | 3p25.3-p24.1 | TRAK1 | 22906 |
| 10 | 3p25 | HRH1 | 3269 |
| 11 | 3p24-p22 | RAB5A | 5868 |
| 12 | 3p24.3 | EFHB | 151651 |
| 13 | 3p24.3 | ZNF659 | 79750 |
| 14 | 3p24.2 | THRB | 7068 |
| 15 | 3p24.2 | NGLY1 | 55768 |
| 16 | 3p24.2 | OXSM | 54995 |
| 17 | 3p24.2 | UBE2E2 | 7325 |
| 18 | 3p24.1 | NEK10 | 152110 |
| 19 | 3p24 | RARB | 5915 |
| 20 | 3p24 | TOP2B | 7155 |
| 21 | 3p24 | LRRC3B | 116135 |
| 22 | 3p23-p22 | ACAA1 | 30 |
| 23 | 3p22-p21.33 | GORASP1 | 64689 |
| 24 | 3p22-p21.3 | GOLGA4 | 2803 |
| 25 | 3p22-p21.3 | DLEC1 | 9940 |
| 26 | 3p22-p21.3 | OXSR1 | 9943 |
| 27 | 3p22-p21.3 | CYP8B1 | 1582 |
| 28 | 3p22-p21 | ZNF35 | 7584 |
| 29 | 3p22.3 | OSBPL10 | 114884 |
| 30 | 3p22.3 | GPD1L | 23171 |
| 31 | 3p22.3 | CMTM7 | 112616 |
| 32 | 3p22.3 | CMTM6 | 54918 |
| 33 | 3p22.3 | DYNC1LI1 | 51143 |
| 34 | 3p22.3 | CRTAP | 10491 |
| 35 | 3p22.3 | FBXL2 | 25827 |
| 36 | 3p22.3 | UBP1 | 7342 |
| 37 | 3p22.2 | TTC21A | 199223 |
| 38 | 3p22.2 | RPSA | 3921 |
| 39 | 3p22.1-p21.33 | TMEM16K | 55129 |
| 40 | 3p22.1 | EPM2AIP1 | 9852 |
| 41 | 3p22.1 | SLC25A38 | 54977 |
| 42 | 3p22.1 | MYRIP | 25924 |
| 43 | 3p22.1 | ULK4 | 54986 |
| 44 | 3p22.1 | SEC22C | 9117 |
| 45 | 3p22.1 | CCDC13 | 152206 |
| 46 | 3p22.1 | HIGD1A | 25994 |
| 47 | 3p22.1 | SNRK | 54861 |
| 48 | 3p22 | SLC4A7 | 9497 |
| 49 | 3p22 | TGFBR2 | 7048 |
| 50 | 3p22 | MYD88 | 4615 |
| 51 | 3p22 | AXUD1 | 64651 |
| 52 | 3p22 | CCR8 | 1237 |
| 53 | 3p22 | VIPR1 | 7433 |
| 54 | 3p21-p14 | WNT5A | 7474 |
| 55 | 3p21\|3p21.3 | CX3CR1 | 1524 |
| 56 | 3p21.3-p21.2 | EOMES | 8320 |
| 57 | 3p21.33 | GLB1 | 2720 |
| 58 | 3p21.33 | WDR48 | 57599 |
| 59 | 3p21.31 | ZNF502 | 91392 |
| 60 | 3p21.31 | ZNF501 | 115560 |
| 61 | 3p21.31 | KIF15 | 56992 |
| 62 | 3p21.3 | MLH1 | 4292 |
| 63 | 3p21.3 | ITGA9 | 3680 |
| 64 | 3p21.3 | CTDSPL | 10217 |
| 65 | 3p21.3 | VILL | 50853 |
| 66 | 3p21.3 | SLC22A13 | 9390 |
| 67 | 3p21.3 | CCBP2 | 1238 |
| 68 | 3p21.1-p12 | ATXN7 | 6314 |
| 69 | 3p21.1 | NPCDR1 | 246734 |
| 70 | 3p21 | ABHD5 | 51099 |
| 71 | 3p14.3 | CACNA1D | 776 |
| 72 | 3p14.3 | C3orf63 | 23272 |
| 73 | 3p14.2 | FHIT | 2272 |
| 74 | 3p14.2 | FEZF2 | 55079 |
| 75 | 3p14.2 | CADPS | 8618 |
| 76 | 3p14.1 | C3orf49 | 132200 |
| 77 | 3p14.1 | THOC7 | 80145 |
| 78 | 3p14 | KBTBD8 | 84541 |
| 79 | 5q33 | MEGF10 | 84466 |
| 80 | 5q31.1-q32 | LECT2 | 3950 |
| 81 | 5q31.1 | TCF7 | 6932 |
| 82 | 5q31.1 | LOC153328 | 153328 |
| 83 | 5q31.1 | IL9 | 3578 |
| 84 | 5q31 | ALDH7A1 | 501 |
| 85 | 5q31 | ADAMTS19 | 171019 |
| 86 | 5q31 | SKP1A | 6500 |
| 87 | 5q31 | FBXL21 | 26223 |
| 88 | 5q23-q31 | FBN2 | 2201 |
| 89 | 5q23.3-q31.1 | LMNB1 | 4001 |
| 90 | 5q23.3 | SLC27A6 | 28965 |
| 91 | 5q23.2 | PPIC | 5480 |
| 92 | 5q23.2 | CCDC100 | 153241 |
| 93 | 5q23.2 | GRAMD3 | 65983 |
| 94 | 5q23.2 | PRRC1 | 133619 |
| 95 | 5q23.2 | RNUXA | 51808 |
| 96 | 5q23.1 | FLJ90650 | 206338 |
| 97 | 5q23.1 | COMMD10 | 51397 |
| 98 | 5q23.1 | SEMA6A | 57556 |
| 99 | 5q23.1 | DTWD2 | 285605 |
| 100 | 5q23.1 | TNFAIP8 | 25816 |
| 101 | 5q23.1 | PRR16 | 51334 |
| 102 | 5q22.3 | TRIM36 | 55521 |
| 103 | 5q22.3 | PGGT1B | 5229 |
| 104 | 5q22.3 | CCDC112 | 153733 |
| 105 | 5q22.2 | EPB41L4A | 64097 |
| 106 | 5q22.2 | YTHDC2 | 64848 |
| 107 | 5q22.1 | TSLP | 85480 |
| 108 | 5q22.1 | WDR36 | 134430 |
| 109 | 5q22 | FEM1C | 56929 |
| 110 | 5q22 | AP3S1 | 1176 |
| 111 | 5q22 | DMXL1 | 1657 |
| 112 | 5q21-q22 | C5orf26 | 114915 |
| 113 | 5q21.3 | PJA2 | 9867 |
| 114 | 5q21 | ST8SIA4 | 7903 |
| 115 | 5q21 | EFNA5 | 1946 |
| 116 | 5q21 | FER | 2241 |
| 117 | 5q21 | HSD17B4 | 3295 |
| 118 | 5q15-q21 | PCSK1 | 5122 |
| 119 | 5q15-q21 | CHD1 | 1105 |
| 120 | 5q15 | C5orf21 | 83989 |
| 121 | 5q15 | MCTP1 | 79772 |
| 122 | 5q15 | FAM81B | 153643 |
| 123 | 5q15 | KIAA0372 | 9652 |
| 124 | 5q15 | CAST | 831 |
| 125 | 5q15 | ARTS-1 | 51752 |
| 126 | 5q15 | LRAP | 64167 |
| 127 | 5q15 | LNPEP | 4012 |
| 128 | 5q15 | LIX1 | 167410 |
| 129 | 5q15 | RIOK2 | 55781 |
| 130 | 5q14-q21 | PAM | 5066 |
| 131 | 5q14.3 | CETN3 | 1070 |
| 132 | 5q14.3 | POLR3G | 10622 |
| 133 | 5q14 | NR2F1 | 7025 |
| 134 | 5q13 | RAB3C | 115827 |
| 135 | 5q12.3 | P18SRP | 285672 |
| 136 | 5q12.3 | SDCCAG10 | 10283 |
| 137 | 5q12.3 | PPWD1 | 23398 |
| 138 | 5q12.3 | TRIM23 | 373 |
| 139 | 5q12.3 | FLJ13611 | 80006 |
| 140 | 5q12.3 | SGTB | 54557 |
| 141 | 5q12.3 | NLN | 57486 |
| 142 | 5q12.3 | ERBB2IP | 55914 |
| 143 | 5q12.3 | SFRS12 | 140890 |
| 144 | 5q12.1 | PART1 | 25859 |
| 145 | 5q12.1 | DEPDC1B | 55789 |
| 146 | 5q12.1 | IPO11 | 51194 |
| 147 | 5q12 | PDE4D | 5144 |
| 148 | 5q12 | CD180 | 4064 |
| 149 | 5q11.2 | C5orf29 | 202309 |

TABLE 3
Marker genes of poor prognosis in early stage lung ADC

| | | Entrez | Hazard | 95% | |
|---|---|---|---|---|---|
| Gene Name | Description | Gene ID | Ratio | CI | p value |
| AP1S1 | Adaptor-related protein | 1174 | 4.59 | 1.77-11.91 | 0.002 |
| | complex 1, sigma 1 subunit | | | | |
| AP4M1 | Adaptor-related protein | 9179 | 3.68 | 1.5-9.01 | 0.004 |
| | complex 4, mu 1 subunit | | | | |
| BRD9 | Bromodomain containing 9 | 65980 | 3.89 | 1.1-13.8 | 0.035 |
| CCDC21 | Coiled-coil domain | 64793 | 6.46 | 1.15-36.14 | 0.034 |
| | containing 21 | | | | |
| CCL8 | Chemokine (C-C motif) | 6355 | 1.74 | 1-3.04 | 0.050 |
| | ligand 8 | | | | |
| COPS6 | COP9 constitutive | 10980 | 2.95 | 1.4-6.22 | 0.004 |
| | photomorphogenic homolog | | | | |
| | subunit 6 (Arabidopsis) | | | | |
| CSDE1 | Cold shock domain | 7812 | 3.61 | 1.1-11.84 | 0.034 |
| | containing E1, RNA-binding | | | | |
| EP300 | E1A binding protein p300 | 2033 | 6.15 | 1.23-30.63 | 0.027 |
| GNB2 | Guanine nucleotide binding | 2783 | 6.69 | 2.1-21.29 | 0.001 |
| | protein (G protein), beta | | | | |
| | polypeptide 2 | | | | |
| HIPK1 | Homeodomain interacting | 204851 | 2.98 | 1.1-8.11 | 0.032 |
| | protein kinase 1 | | | | |
| HRSP12 | Heat-responsive protein 12 | 10247 | 2.98 | 1.19-7.45 | 0.020 |
| LAPTM4B | Lysosomal associated protein | 55353 | 1.47 | 1.01-2.13 | 0.044 |
| | transmembrane 4 beta | | | | |
| MCM7 | MCM7 minichromosome | 4176 | 2.74 | 1.45-5.19 | 0.002 |
| | maintenance deficient 7 (S. cerevisiae) | | | | |
| MGC4677 | Hypothetical protein | 112597 | 3.97 | 1.89-8.31 | <0.001 |
| | MGC4677 | | | | |
| OLFM2 | Olfactomedin 2 | 93145 | 4.45 | 1.5-13.15 | 0.007 |
| PDCD6 | Programmed cell death | 10016 | 4.94 | 1.22-8.52 | 0.02 |
| | protein 6 | | | | |
| POP7 | Processing of precursor 7, | 10248 | 3.25 | 1.2-8.79 | 0.020 |
| | ribonuclease P subunit (S. cerevisiae) | | | | |
| PPA1 | Pyrophosphatase (inorganic) 1 | 5464 | 3.06 | 1.31-7.16 | 0.010 |
| RABL4 | RAB, member of RAS | 11020 | 3.88 | 1.12-13.43 | 0.032 |
| | oncogene family-like 4 | | | | |
| RPL30 | Ribosomal protein L30 | 6156 | 19.70 | 2.44-158.85 | 0.005 |
| SERPINE1 | Serpin peptidase inhibitor, | 5054 | 3.43 | 1.84-6.4 | <0.001 |
| | clade E (nexin, plasminogen | | | | |
| | activator inhibitor type 1), | | | | |
| | member 1 | | | | |
| SH3BGRL3 | SH3 domain binding glutamic | 83442 | 5.01 | 1.75-14.35 | 0.003 |
| | acid-rich protein like 3 | | | | |
| SLC25A17 | Solute carrier family 25 | 10478 | 3.68 | 1.32-10.26 | 0.013 |
| | (mitochondrial carrier), | | | | |
| | member 17 | | | | |
| ST13 | Suppression of tumorigenicity | 6767 | 2.02 | 1.21-3.37 | 0.007 |
| | 13 | | | | |
| TAF6 | TAF6 RNA polymerase II, | 6878 | 3.99 | 1.52-10.52 | 0.005 |
| | TATA box binding protein | | | | |
| | (TBP)-associated factor, | | | | |
| | 80 kDa | | | | |
| TLE3 | Transducin-like enhancer of | 7090 | 5.09 | 1.45-17.83 | 0.011 |
| | split 3 (E(sp1) homolog, | | | | |
| | Drosophila) | | | | |
| TOB2 | Transducer of ERBB2, 2 | 10766 | 3.37 | 1.01-11.32 | 0.049 |
| ZNF561 | Zinc finger protein 561 | 93134 | 8.20 | 1.41-47.74 | 0.019 |

Identified in silico as poor prognosis markers in early stage (stage 1) ADC in the ‘Duke’ microarray expression dataset (19) or from NSCLC samples from University Health Network (59).

TABLE 4
Marker genes of poor prognosis in early stage lung ADC

| Gene | Genomic | Entrez | Hazard | p |
|---|---|---|---|---|
| Name | location | Gene | Ratio | value |
| C5orf21 | 5q15 | 83989 | 0.14 | 0.038 |
| C5orf29 | 5q11.2 | 202309 | 0.00 | 0.003 |
| CACNA1D | 3p14.3 | 776 | 0.19 | 0.024 |
| CCDC13 | 3p22.1 | 152206 | 0.44 | 0.034 |
| CNTN6 | 3p26-p25 | 27255 | 0.02 | 0.030 |
| CRTAP | 3p22.3 | 10491 | 0.00 | 0.000 |
| DMXL1 | 5q22 | 1657 | 0.12 | 0.041 |
| EOMES | 3p21.3-p21.2 | 8320 | 0.29 | 0.044 |
| ERBB2IP | 5q12.3 | 55914 | 0.01 | 0.009 |
| FEZF2 | 3p14.2 | 55079 | 0.01 | 0.018 |
| HRH1 | 3p25 | 3269 | 0.11 | 0.016 |
| LRAP | 5q15 | 64167 | 0.00 | 0.001 |
| MEGF10 | 5q33 | 84466 | 0.04 | 0.006 |
| NPCDR1 | 3p21.1 | 246734 | 0.01 | 0.022 |
| PAM | 5q14-q21 | 5066 | 0.06 | 0.012 |
| PPWD1 | 5q12.3 | 23398 | 0.01 | 0.041 |
| RAB5A | 3p24-p22 | 5868 | 0.06 | 0.049 |
| SEMA6A | 5q23.1 | 57556 | 0.03 | 0.006 |
| SFRS12 | 5q12.3 | 140890 | 0.02 | 0.034 |
| SNRK | 3p22.1 | 54861 | 0.00 | 0.024 |
| TRIM36 | 5q22.3 | 55521 | 0.07 | 0.035 |
| TTC21A | 3p22.2 | 199223 | 0.05 | 0.041 |
| ULK4 | 3p22.1 | 54986 | 0.02 | 0.001 |
| VIPR1 | 3p22 | 7433 | 0.09 | 0.002 |
| ZNF502 | 3p21.31 | 91392 | 0.14 | 0.029 |

TABLE 5
Correlative gene expression validation results
for the candidate amplified marker genes

| | Toronto | Harvard (17) | Michigan (18) |
|---|---|---|---|
| No. of ADC samples | 39 | 127 | 86 |
| No. of normal lung | 10 | 17 | 10 |
| samples | | | |
| Array type | U133A | U95A | HuGeneFL |
| No. of genes/ProbeSets | 13840/22215 | 9513/12,625 | 5945/7129 |
| No. of genes/ProbeSets | 4885/6251 | 3175/3635 | 1692/1909 |
| upregulated | | | |
| No. of genes/ProbeSets | 3076/3998 | 1887/2119 | 929/946 |
| downregulated | | | |
| % of genes upregulated | 35.29 | 33.37 | 28.46 |
| % FDR used | 4.06 | 3.98 | 4.98 |
| No. of genes from 113 | 87 | 59 | 42 |
| gene list that are present | | | |
| on array | | | |
| No. of upregulated genes | 38 | 22 | 15 |
| that match 113 gene list | | | |
| No. of genes that match | 1.54 | 0.88 | 0.75 |
| by mistake based on | | | |
| FDR | | | |
| Expected no. of | 30 | 19 | 12 |
| upregulated genes based | | | |
| on observed % of | | | |
| upregulated genes | | | |
| Validation rate % | 41.90 | 35.80 | 33.93 |

TABLE 6
Correlative gene expression validation results
for the candidate deleted marker genes

| | Toronto | Harvard (57) | Michigan (58) |
|---|---|---|---|
| No. of ADC samples | 39 | 127 | 86 |
| No. of normal lung | 10 | 17 | 10 |
| samples | | | |
| Array type | U133A | U95A | HuGeneFL |
| No. of genes/ProbeSets | 13840/22215 | 9513/12,625 | 5945/7129 |
| No. of genes/ProbeSets | 3076/3998 | 1887/2119 | 929/946 |
| downregulated | | | |
| No. of genes/ProbeSets | 4885/6251 | 3175/3635 | 1692/1909 |
| upregulated | | | |
| % of genes | 22.22 | 19.83 | 15.62 |
| downregulated | | | |
| % FDR used | 4.06 | 3.98 | 4.98 |
| No. of genes from 149 | 113 | 84 | 48 |
| gene list that are present | | | |
| on array | | | |
| No. of downregulated | 53 | 23 | 10 |
| genes that match 149 | | | |
| gene list | | | |
| No. of genes that match | 2.15 | 0.9154 | 0.498 |
| by mistake based on | | | |
| FDR | | | |
| Expected no. of | 25 | 16 | 7 |
| downregulated genes | | | |
| based on observed % | | | |
| of downregulated genes | | | |
| Validation rate % | 45.00 | 26.29 | 19.80 |

TABLE 7
Clinical and demographic information of the various validation datasets

| | | QPCR | | Duke ADC | |
|---|---|---|---|---|---|
| | | | Stage I-II | | Stage I |
| Dataset | | All | ADC | All | ADC |
| No. | | 85 | 47 | 89 | 34 |
| Histology | ADC | 54 | 47 | 43 | 34 |
| | SCC | 26 | 0 | 46 | 0 |
| | Others* | 5 | 0 | 0 | 0 |
| Stage | I | 61 | 38 | 67 | 34 |
| | II | 15 | 9 | 18 | 0 |
| | III | 9 | 0 | 4 | 0 |
| Gender | Male | 35 | 22 | 54 | 17 |
| | Female | 42 | 18 | 35 | 17 |
| | Unknown | 8 | 7 | 0 | 0 |
| Age - | | 70.9 | 70.6 | 65.1 | 64.8 |
| mean | | (45.6-84.9) | (45.6-84.9) | (32.0- | (43.0- |
| (range) | | | | 83.0) | 83.0) |

ADC—Adenocarcinoma;
SCC—Squamous cell carcinoma.
Others* - Large cell carcinoma and adenosquamous carcinoma.

TABLE 8
Clinical characteristics of the tumors studied by array CGH

| | | | AWBF in | AWBF in |
|---|---|---|---|---|
| Characteristic | Category | BAC | BAC area | invasive area |
| T | T1 | 10 | 9 | 3 |
| | T2 | 4 | 2 | 1 |
| N | N0 | 13 | 10 | 4 |
| | N1 | 0 | 1 | 0 |
| | N2 | 1 | 0 | 0 |
| Stage | 1A | 7 | 7 | 3 |
| | 1B | 4 | 1 | 1 |
| | N/A | 3 | 3 | 0 |
| Gender | F | 11 | 7 | 2 |
| | M | 3 | 4 | 2 |
| Follow up | Range (years) | 0.2-7.0 | 0.6-7.2 | 0.6-6.0 |
| | Median (years) | 2 | 1.9 | 2.4 |
| Status | Alive | 10 | 9 | 3 |
| | Unknown | 4 | 2 | 1 |

N/A - Staging is not applicable due to synchronous tumors.

TABLE 9
The Toronto DNA microarray dataset

| | Number of | |
|---|---|---|
| | patients | Sub group |
| Histology | ADC | 39 |
| Gender | Male | 22 |
| | Female | 17 |
| Age | Range | 39.6-78.9 |
| | Median | 64.5 |
| Stage | 1A | 10 |
| | 1B | 18 |
| | 2A | 2 |
| | 2B | 9 |

TABLE 10
Primer sequences for amplified genes

| No. | Gene | Forward | Reverse |
|---|---|---|---|
| 1 | AP1S1 | TGGCTTTTATTCCCTGCC | ACAGTTATTAGGGAGG |
| | | TTT | GAAGGACAT |
| | | (SEQ ID NO: 1) | (SEQ ID NO: 2) |
| 2 | AP4M1 | CGCCTATGTCATTCGGAT | GTGGGACAAACTGCCA |
| | | CTG | CCTT |
| | | (SEQ ID NO: 3) | (SEQ ID NO: 4) |
| 3 | BRD9 | CGACCCCTATGAGTTTCT | GCTGAAGGTGGTCTAG |
| | | TCAGTCT | AGTTAGGTCTT |
| | | (SEQ ID NO: 5) | (SEQ ID NO: 6) |
| 4 | CCDC21 | GCGTCGTGACATTGAGGA | CCCATGTCCTGGGCAT |
| | | CTT | ATCT |
| | | (SEQ ID NO: 7) | (SEQ ID NO: 8) |
| 5 | CCL8 | CATGAAGCATCTGGACCA | GCTCTGACTCTCAGTC |
| | | AATATT | CATGTATGA |
| | | (SEQ ID NO: 9) | (SEQ ID NO: 10) |
| 6 | COPS6 | TGCTGTTTGCTGAGCTGA | TTCGGGCTACGTGGTC |
| | | CCTA | TACAC |
| | | (SEQ ID NO: 11) | (SEQ ID NO: 12) |
| 7 | CSDE1 | TCAGGATGGCATTGAGCT | CCAGTGCGCTGATTAA |
| | | ACAG | GAATCA |
| | | (SEQ ID NO: 13) | (SEQ ID NO: 14) |
| 8 | EP300 | GCAAAAATAAGAGCAGCC | TGTGAGAGGTCGTTAG |
| | | TGAGTA | ATACATTGG |
| | | (SEQ ID NO: 15) | (SEQ ID NO: 16) |
| 9 | GNB2 | CGCTCCTCCTGGGTAATG | GCTGTAGATGGAGCAG |
| | | AC | ATGTTGTC |
| | | (SEQ ID NO: 17) | (SEQ ID NO: 18) |
| 10 | HIPK1 | CCTGCTCAGTACCAACAC | ATTGTTGAGCCTCGGG |
| | | CAGTT | AAGAC |
| | | (SEQ ID NO: 19) | (SEQ ID NO: 20) |
| 11 | HRSP12 | TGTGAGTTTGATGAAAAT | AACTGGATTCCTTCTC |
| | | ATCTGAAGCT | TAATATTCATGTT |
| | | (SEQ ID NO: 21) | (SEQ ID NO: 22) |
| 12 | LAPTM4B | CTGAGGGCAGCAGCTTGA | AGGCTCATGGCAAAAG |
| | | CT | TGAAAT |
| | | (SEQ ID NO: 23) | (SEQ ID NO: 24) |
| 13 | MCM7 | ATGTCTGGCAGGTCAATG | GCAGGCTGGAATCAGA |
| | | CTT | CAAAA |
| | | (SEQ ID NO: 25) | (SEQ ID NO: 26) |
| 14 | MGC4677 | CCTCCAGCACCTCTACCT | AGGGAATCTTTCAGCT |
| | | GTTG | GCATTC |
| | | (SEQ ID NO: 27) | (SEQ ID NO: 28) |
| 15 | OLFM2 | TTTACCAACACGTCCAGT | CGAGATGTGGGAATAC |
| | | TACGA | TGGTTGT |
| | | (SEQ ID NO: 29) | (SEQ ID NO: 30) |
| 16 | POP7 | ACACACGGGAGCCACTGA | TTGGGTGTGACCCTGA |
| | | CT | AGACT |
| | | (SEQ ID NO: 31) | (SEQ ID NO: 32) |
| 17 | PPA1 | TCCATCACCAGAAAAACT | TCCAGATGAACACGAT |
| | | AATGAGAT | GTAGCAATA |
| | | (SEQ ID NO: 33) | (SEQ ID NO: 34) |
| 18 | RABL4 | CAAGCAGTTCCACCAGCT | GATCTGCTCCAGCTCG |
| | | GTAC | TCATG |
| | | (SEQ ID NO: 35) | (SEQ ID NO: 36) |
| 19 | RPL30 | ATGTTGGCTAAAACTGGT | CACTCTGTAGTATTTT |
| | | GTCCAT | CCGCATGCT |
| | | (SEQ ID NO: 37) | (SEQ ID NO: 38) |
| 20 | SERPINE1 | GGCTGGTGACAGGCCAAA | CAGTGCCACAGTGGAC |
| | | (SEQ ID NO: 39) | TCTGA |
| | | | (SEQ ID NO: 40) |
| 21 | SH3BGRL3 | GCTGGACTCCATCACCAC | AGTTGGTCAAAAGGTC |
| | | ACT | CTTCATG |
| | | (SEQ ID NO: 41) | (SEQ ID NO: 42) |
| 22 | SLC25A17 | GAAGCGTGCACACCAACA | CCTCTTGAGCATCTTC |
| | | CT | GGAATT |
| | | (SEQ ID NO: 43) | (SEQ ID NO: 44) |
| 23 | ST13 | GATCACCTTATGGATGTC | ACCCCAGCTCTCTTGA |
| | | GCAAT | TGAGAAG |
| | | (SEQ ID NO: 45) | (SEQ ID NO: 46) |
| 24 | TAF6 | CTCAGCCTGCTCCGTGAT | GCACGTGTGTACATGT |
| | | G | CTGCAT |
| | | (SEQ ID NO: 47) | (SEQ ID NO: 48) |
| 25 | TLE3 | CTCGTCTGTCTTGAGTTG | TCTTGTCACCAGAGCC |
| | | TGACATT | TGTTACA |
| | | (SEQ ID NO: 49) | (SEQ ID NO: 50) |
| 26 | TOB2 | TCCCTTTGAGGTGTCCTA | GTCATCCAGGTACAGC |
| | | CCA | ACTTTCAC |
| | | (SEQ ID NO: 51) | (SEQ ID NO: 52) |
| 27 | ZNF561 | TCAGGGAACAGTTTTGCC | CCCTCAGAAGTATTCC |
| | | TTAAG | CTCCATT |
| | | (SEQ ID NO: 53) | (SEQ ID NO: 54) |
| 28 | EPO | CAGCCTGTCCCATGGACA | TCCTCTGGCCCCTGAG |
| | | CT | ATG |
| | | (SEQ ID NO: 105) | (SEQ ID NO: 106) |
| 29 | TERT | TTCAAGGCTGGGAGGAAC | TGACACTTCAGCCGCA |
| | | AT | AGAC |
| | | (SEQ ID NO: 107) | (SEQ ID NO: 108) |
| 30 | PDCD6 | CTCGTGAAGAGCAGCACA | CTGTGCTCCATTCCCT |
| | | ACAT | CACA |
| | | (SEQ ID NO: 109) | (SEQ ID NO: 110) |
| 31 | PLGLB2 | TGCAAGACTGGGAATGGA | CAGGTGATGCCATTTT |
| | | AAG | TTGTTTT |
| | | (SEQ ID NO: 111) | (SEQ ID NO: 112) |
| 32 | SAR1A | CGTGCTGGTGCAGGTCAG | CATACAGCTTTACCCG |
| | | T | GCAAAT |
| | | (SEQ ID NO: 113) | (SEQ ID NO: 114) |
| 33 | SYCP1 | TTCTTATTTGCCAGAGCC | TCCAGACAATATGTAG |
| | | AAATT | TTAACATTTAGGCTAA |
| | | (SEQ ID NO: 115) | (SEQ ID NO: 116) |

TABLE 11
Primer sequences for deleted genes

| No. | Gene | Forward | Reverse |
|---|---|---|---|
| 1 | C5orf21 | CGCTCTCAACTGGAGCA | GCCCTCAGGGAAAAAC |
| | | AACT | CTAGTG |
| | | (SEQ ID NO: 55) | (SEQ ID NO: 56) |
| 2 | C5orf29 | TCTTAGTGGTTTGTGGA | CGGTAAGGTAAATCGT |
| | | ATTGGGT | GTGGC |
| | | (SEQ ID NO: 57) | (SEQ ID NO: 58) |
| 3 | CACNA1D | CCTGCTTGGAAACCATG | GGGTGGTATTGGTCTG |
| | | TCA | CTGAAG |
| | | (SEQ ID NO: 59) | (SEQ ID NO: 60) |
| 4 | CCDC13 | ACCCCAGAGGTGAAGGC | TGGTTTCGGAGGTCAC |
| | | C | TCATCT |
| | | (SEQ ID NO: 61) | (SEQ ID NO: 62) |
| 5 | CNTN6 | TTTACTCAGGAGCCACA | GGTAACCATTAGCAGC |
| | | TGATGTC | ACAATTCA |
| | | (SEQ ID NO: 63) | (SEQ ID NO: 64) |
| 6 | CRTAP | AATGATGAAGAGGAACA | TGGTTTCCAGGTCTTT |
| | | TGGCATA | AATGTAGTCC |
| | | (SEQ ID NO: 65) | (SEQ ID NO: 66) |
| 7 | DMXL1 | AAATTGAGACAGGGCCT | CAGTATTCTCATTTTC |
| | | GCA | ATTGTTCCATC |
| | | (SEQ ID NO: 67) | (SEQ ID NO: 68) |
| 8 | EOMES | ACCCCCTTCCATCAAAT | GCCGCCTTCGCTTACA |
| | | CTCTAG | AG |
| | | (SEQ ID NO: 69) | (SEQ ID NO: 70) |
| 9 | ERBB2IP | CAGTGATAGAGAATTGC | CTTTACACACTGGCAG |
| | | TGTGGAGA | GCCTC |
| | | (SEQ ID NO: 71) | (SEQ ID NO: 72) |
| 10 | FEZF2 | AACCTAACCTTCCATAT | AAACCCTTTGCCGCAA |
| | | GCACACC | GTG |
| | | (SEQ ID NO: 73) | (SEQ ID NO: 74) |
| 11 | HRH1 | TTCTGGAATCCAAACCA | CTGCTGTTCTTCTATG |
| | | CAGTCTTA | GTGCCTAA |
| | | (SEQ ID NO: 75) | (SEQ ID NO: 76) |
| 12 | LRAP | ACCCCGTCTCCGCTAAA | CCTGCCGAGTAGCTGG |
| | | AAT | GAC |
| | | (SEQ ID NO: 77) | (SEQ ID NO: 78) |
| 13 | MEGF10 | ATGCAAGTGTGAATTAC | ACGGGCAGTTTGCTGT |
| | | GTGGTATG | ACATC |
| | | (SEQ ID NO: 79) | (SEQ ID NO: 80) |
| 14 | NPCDR1 | TGTCGAGCATTCTCTTT | CCCCTTTCTGTAGGAT |
| | | CTCTGTT | TCCCTT |
| | | (SEQ ID NO: 81) | (SEQ ID NO: 82) |
| 15 | PAM | CTTTGGTGCCTTTCCTG | TGTCGTCATGTAGCAC |
| | | TTCAG | AAAGTTTCT |
| | | (SEQ ID NO: 83) | (SEQ ID NO: 84) |
| 16 | PPWD1 | GAGTGACTAAAGGAATG | GGGCTTATCTGTTTTG |
| | | GAAGTTGTACA | GGATTG |
| | | (SEQ ID NO: 85) | (SEQ ID NO: 86) |
| 17 | RAB5A | ATGGGATACAGCTGGTC | TGGCTGCTTGTGCTCC |
| | | AAGAAC | TCT |
| | | (SEQ ID NO: 87) | (SEQ ID NO: 88) |
| 18 | SEAM6A | CATCACCGTCTACTGCG | GCTCCTTCTCCTTGCG |
| | | TCTGT | CTG |
| | | (SEQ ID NO: 89) | (SEQ ID NO: 90) |
| 19 | SFRS12 | CTGCACTCAATGCTGGA | AGATTTCTTTCCTGTT |
| | | ATCAA | TACATCTTGTTG |
| | | (SEQ ID NO: 91) | (SEQ ID NO: 92) |
| 20 | SNRK | GCTACAGAGAGATCCCA | GGTCCACTCCCTGAAG |
| | | AGAGAAGG | CCA |
| | | (SEQ ID NO: 93) | (SEQ ID NO: 94) |
| 21 | TRIM36 | GAGACGAGGCTACCGCT | CACTTTTATTTCCTGT |
| | | GC | TTGGGAGAA |
| | | (SEQ ID NO: 95) | (SEQ ID NO: 96) |
| 22 | TTC21A | CAGCCTGAAGGAAATAC | TGAGCCAGAGGAAAAG |
| | | GCAA | GCC |
| | | (SEQ ID NO: 97) | (SEQ ID NO: 98) |
| 23 | ULK4 | AGCAACTTTTGCTTGGC | CACCTCCTCCTTCCTC |
| | | AAAA | TGCTG |
| | | (SEQ ID NO: 99) | (SEQ ID NO: 100) |
| 24 | VIPR1 | TCCTGAATTCCCCTTGC | AGATGATACATGAGAT |
| | | CA | GGAGGCC |
| | | (SEQ ID NO: 101) | (SEQ ID NO: 102) |
| 25 | ZNF502 | GCATCTGGGAATACAGG | GGGAGACAGTGGCAAT |
| | | GTAGAA | GTGTAG |
| | | (SEQ ID NO: 103) | (SEQ ID NO: 104) |

TABLE 12
The study group of copy numbers of 33 genes by QPCR on genomic DNA

| TERT/ | array | | |
|---|---|---|---|
| PDCD6 and | CGH | Cases sampled | |
| other genes | performed | in 2 areas | Comment |
| BAC | 12 | 2 | 2 cases had synchronous |
| | | | tumors: BAC and AWBF. |
| AWBF in BAC | 11 | 2 + 3 | 2 cases had synchronous |
| area | | | tumors: BAC and AWBF. |
| | | | 3 cases were sampled in |
| | | | both BAC and invasive |
| | | | area |
| AWBF in | 4 | 3 | 3 cases were sampled in |
| invasive area | | | both BAC and invasive |
| | | | area |
| Total | 27 | 5 | 27 − 5 = 22 |

Total number of cases used: 22
Two array CGH samples from the BAC group were not evaluated by QPCR due to technical problems.
The 33 genes evaluated by QPCR included (Table 13):
a. The 28 genes found to be markers of poor prognosis in early stage lung ADC out of the 113 amplified genes (Table 3).
b. The following genes: TERT, PLGLB2, EPO, SYCP1 and SAR1A were amplified but not prognostic at expression level.

TABLE 13
Ranking of Diagnostic Biomarkers with Differentially Increased Gene Copy Number in AWBF

| | | | BAC vs | | Optimal | | | |
|---|---|---|---|---|---|---|---|---|
| | | | AWBF | ROC | QPCR | | | |
| | | Chromosome | QPCR | Analysis | Fold Change | Relative | | |
| Gene | Accession | Number | p-value | (AUC) | Threshold | Risk | Sensitivity | Specificity |
| EPO | NM_000799 | 7 | 0.002 | 0.870 | 1.2 | 7.2 | 0.600 | 0.917 |
| PPA1 | NM_021129 | 10 | 0.020 | 0.780 | 1.2 | 6.4 | 0.533 | 0.917 |
| SERPINE1 | NM_000602 | 7 | 0.006 | 0.832 | 1.3 | inf | 0.533 | 1.000 |
| SLC25A17 | NM_006358 | 22 | 0.010 | 0.804 | 1.3 | 5.6 | 0.467 | 0.917 |
| PDCD6 | NM_013232 | 5 | 0.021 | 0.783 | 1.3 | 6.4 | 0.533 | 0.917 |
| SAR1A | NM_020150 | 10 | 0.030 | 0.769 | 1.3 | 6.4 | 0.533 | 0.917 |
| AP1S1 | NM_001283 | 7 | 0.030 | 0.766 | 1.3 | inf | 0.400 | 0.917 |
| MCM7 | NM_005916 | 7 | 0.030 | 0.766 | 1.3 | 7.2 | 0.600 | 0.917 |
| COPS6 | NM_006833 | 7 | 0.020 | 0.764 | 1.3 | 8.0 | 0.667 | 0.917 |
| PLGLB2 | NM_002665 | 2 | 0.080 | 0.720 | 1.3 | 4.8 | 0.400 | 0.917 |
| POP7 | NM_005837 | 7 | 0.020 | 0.801 | 1.4 | inf | 0.600 | 1.000 |
| HIPK1 | NM_152696 | 1 | 0.020 | 0.780 | 1.4 | 5.6 | 0.467 | 0.917 |
| TOB2 | NM_016272 | 22 | 0.030 | 0.766 | 1.4 | 6.4 | 0.533 | 0.917 |
| TLE3 | NM_005078 | 15 | 0.230 | 0.648 | 1.4 | 5.6 | 0.467 | 0.917 |
| EP300 | NM_001429 | 22 | 0.016 | 0.780 | 1.5 | 6.4 | 0.533 | 0.917 |
| CCDC21 | NM_022778 | 1 | 0.020 | 0.768 | 1.5 | 6.4 | 0.533 | 0.917 |
| CSDE1 | NM_007158 | 1 | 0.038 | 0.768 | 1.5 | 4.0 | 0.333 | 0.917 |
| ST13 | NM_003932 | 22 | 0.040 | 0.754 | 1.5 | 5.6 | 0.467 | 0.917 |
| TERT | NM_003219 | 5 | 0.030 | 0.708 | 1.5 | 6.4 | 0.533 | 0.917 |
| MGC4677 | NM_052871 | 2 | 0.054 | 0.748 | 1.6 | 3.2 | 0.267 | 0.917 |
| ZNF561 | NM_152289 | 19 | 0.040 | 0.743 | 1.6 | 4.0 | 0.333 | 0.917 |
| TAF6 | NM_005641 | 7 | 0.030 | 0.738 | 1.6 | 5.6 | 0.467 | 0.917 |
| BRD9 | NM_023924 | 5 | 0.030 | 0.722 | 1.6 | 6.4 | 0.533 | 0.917 |
| RPL30 | NM_000989 | 8 | 0.010 | 0.791 | 1.7 | 4.8 | 0.400 | 0.917 |
| SH3BGRL3 | NM_031286 | 1 | 0.040 | 0.745 | 1.7 | 4.8 | 0.400 | 0.917 |
| GNB2 | NM_005273 | 7 | 0.040 | 0.744 | 1.7 | 6.4 | 0.533 | 0.917 |
| AP4M1 | NM_004722 | 7 | 0.080 | 0.735 | 1.7 | inf | 0.400 | 1.000 |
| CCL8 | NM_005623 | 17 | 0.040 | 0.735 | 1.7 | 4.0 | 0.333 | 0.917 |
| RABL4 | NM_006860 | 22 | 0.110 | 0.701 | 1.7 | 2.4 | 0.200 | 0.917 |
| HRSP12 | NM_005836 | 8 | 0.120 | 0.690 | 1.8 | 3.2 | 0.267 | 0.917 |
| OLFM2 | NM_058164 | 19 | 0.100 | 0.674 | 1.8 | 2.4 | 0.200 | 0.917 |
| SYCP1 | NM_003176 | 1 | 0.030 | 0.761 | >1.8 | | | |
| LAPTM4B | NM_018407 | 8 | 0.250 | 0.660 | >1.8 | | | |

1. A method of classifying a subject with lung adenocarcinoma, comprising the steps:
(a) determining the expression level of one or more biomarkers in a test sample from the subject, wherein the one or more biomarkers are selected from Table 1, 2, 3 and/or 4;
(b) comparing the expression of the one or more biomarkers with a control, and
(c) classifying the subject with lung adenocarcinoma into a bronchioloalveolar carcinoma (BAC) group or invasive adenocarcinoma (ADC) group or a poor survival group or a good survival group according to a difference or a similarity in the expression of the one or more biomarkers between the control and the test sample.

2. The method of claim 1 for classifying or prognosing a subject with lung adenocarcinoma, comprising the steps:
a) obtaining a subject biomarker expression profile in a sample of the subject;
b) obtaining one or more biomarker reference expression profiles associated with a disease subtype, wherein the subject biomarker expression profile and the biomarker reference profile each has a plurality of values, each value representing an expression level of a biomarker, wherein the one or more biomarkers are selected from Table 1, 2, 3 and/or 4; and
c) selecting the biomarker reference expression profile most similar to the subject biomarker expression profile, to thereby classify the subject as having BAC or invasive ADC, or to thereby predict a prognosis for the subject.

3.-5. (canceled)

6. The method of claim 1, wherein the invasive ADC comprises ADC with BAC features (AWBF).

7.-32. (canceled)

33. The method of claim 1, wherein determining the biomarker expression level or obtaining the expression profile comprises use of quantitative PCR, an array, a DNA microarray, immunohistochemistry or an antibody.

34-39. (canceled)

40. A method of classifying a subject with lung adenocarcinoma, comprising the steps:
a) determining a genomic profile comprising detecting genomic alterations in a test sample,
b) comparing the genomic profile with one or more controls; and
c) classifying the subject as having BAC or invasive ADC according to a difference or similarity in the genomic profile between the one or more controls and the test sample, wherein one of the one or more controls comprises a BAC or BAC with focal areas suspicious for invasion sample and wherein increased genomic alterations compared to the control, classifies the subject as having invasive ADC.

41. (canceled)

42. The method of claim 40 wherein the genomic alterations comprise genome copy gains on one or more of 1p, 2q, 5p, 7p, 11p, 11q, 12q, 16p, 16q, 17q, 20q and/or 21q.

43. (canceled)

44. The method of claim 42 wherein the genome copy gain comprises genes falling within the genomic region 5pter-p15.2.

45. (canceled)

46. The method of claim 42 wherein the genome copy gain comprises TERT, and classifies the subject as having invasive ADC or wherein the genome copy gain comprises EPO, SLC25A17 POP7, PDCD6, SERPINE1, GNB2 and/or ST13 and prognose the subject into the poor survival group.

47.-56. (canceled)

57. The method of claim 40 for classifying a subject with lung adenocarcinoma, comprising the steps:
a) determining a gene copy number profile in a test sample of a subject wherein the gene copy number profile comprises one or more gene copy gains of one or more genes listed in Table 1 or 3;
b) comparing the gene copy number profile to one or more control reference profiles, and
c) classifying the subject into a BAC group or an invasive ADC group according to a difference or a similarity in the gene copy number profile between the test sample and the one or more controls.

58-67. (canceled)

68. The method of claim 40 wherein the step of determining the genome profile comprises FISH analysis.

69. A method of selecting a treatment for a subject with adenocarcinoma, comprising the steps:
a) classifying the subject into a BAC group or an invasive ADC group according to the method of claim 40 wherein each group is associated with a treatment;
b) selecting the treatment associated with the group comprising the subject.

70.-75. (canceled)

76. An array to diagnose, classify or prognose a subject with ADC according to the method of claim 1 comprising for each gene in a plurality of genes, the plurality of genes being at least 1, 2, 3, 4 or more of the genes listed in Tables 1, 2, 3, 4 and/or 13 one or more polynucleotide probes complementary and hybridizable to an expression product of the gene.

77.-100. (canceled)


SEQUENCE LISTING (116 sequences)

SEQ ID NO: 1 - DNA, 21 bp, Homo sapiens
tggcttttattccctgccttt

SEQ ID NO: 2 - DNA, 25 bp, Homo sapiens
acagttattagggagggaaggacat

SEQ ID NO: 3 - DNA, 21 bp, Homo sapiens
cgcctatgtcattcggatctg

SEQ ID NO: 4 - DNA, 20 bp, Homo sapiens
gtgggacaaactgccacctt

SEQ ID NO: 5 - DNA, 25 bp, Homo sapiens
cgacccctatgagtttcttcagtct

SEQ ID NO: 6 - DNA, 27 bp, Homo sapiens
gctgaaggtggtctagagttaggtctt

SEQ ID NO: 7 - DNA, 21 bp, Homo sapiens
gcgtcgtgacattgaggactt

SEQ ID NO: 8 - DNA, 20 bp, Homo sapiens
cccatgtcctgggcatatct

SEQ ID NO: 9 - DNA, 24 bp, Homo sapiens
catgaagcatctggaccaaatatt

SEQ ID NO: 10 - DNA, 25 bp, Homo sapiens
gctctgactctcagtccatgtatga

SEQ ID NO: 11 - DNA, 22 bp, Homo sapiens
tgctgtttgctgagctgaccta

SEQ ID NO: 12 - DNA, 21 bp, Homo sapiens
ttcgggctacgtggtctacac

SEQ ID NO: 13 - DNA, 22 bp, Homo sapiens
tcaggatggcattgagctacag

SEQ ID NO: 14 - DNA, 22 bp, Homo sapiens
ccagtgcgctgattaagaatca

SEQ ID NO: 15 - DNA, 24 bp, Homo sapiens
gcaaaaataagagcagcctgagta

SEQ ID NO: 16 - DNA, 25 bp, Homo sapiens
tgtgagaggtcgttagatacattgg

SEQ ID NO: 17 - DNA, 20 bp, Homo sapiens
cgctcctcctgggtaatgac

SEQ ID NO: 18 - DNA, 24 bp, Homo sapiens
gctgtagatggagcagatgttgtc

SEQ ID NO: 19 - DNA, 23 bp, Homo sapiens
cctgctcagtaccaacaccagtt

SEQ ID NO: 20 - DNA, 21 bp, Homo sapiens
attgttgagcctcgggaagac

SEQ ID NO: 21 - DNA, 28 bp, Homo sapiens
tgtgagtttgatgaaaatatctgaagct

SEQ ID NO: 22 - DNA, 29 bp, Homo sapiens
aactggattccttctctaatattcatgtt

SEQ ID NO: 23 - DNA, 20 bp, Homo sapiens
ctgagggcagcagcttgact

SEQ ID NO: 24 - DNA, 22 bp, Homo sapiens
aggctcatggcaaaagtgaaat

SEQ ID NO: 25 - DNA, 21 bp, Homo sapiens
atgtctggcaggtcaatgctt

SEQ ID NO: 26 - DNA, 21 bp, Homo sapiens
gcaggctggaatcagacaaaa

SEQ ID NO: 27 - DNA, 22 bp, Homo sapiens
cctccagcacctctacctgttg

SEQ ID NO: 28 - DNA, 22 bp, Homo sapiens
agggaatctttcagctgcattc

SEQ ID NO: 29 - DNA, 23 bp, Homo sapiens
tttaccaacacgtccagttacga

SEQ ID NO: 30 - DNA, 23 bp, Homo sapiens
cgagatgtgggaatactggttgt

SEQ ID NO: 31 - DNA, 20 bp, Homo sapiens
acacacgggagccactgact

SEQ ID NO: 32 - DNA, 21 bp, Homo sapiens
ttgggtgtgaccctgaagact

SEQ ID NO: 33 - DNA, 26 bp, Homo sapiens
tccatcaccagaaaaactaatgagat

SEQ ID NO: 34 - DNA, 25 bp, Homo sapiens
tccagatgaacacgatgtagcaata

SEQ ID NO: 35 - DNA, 22 bp, Homo sapiens
caagcagttccaccagctgtac

SEQ ID NO: 36 - DNA, 21 bp, Homo sapiens
gatctgctccagctcgtcatg

SEQ ID NO: 37 - DNA, 24 bp, Homo sapiens
atgttggctaaaactggtgtccat

SEQ ID NO: 38 - DNA, 25 bp, Homo sapiens
cactctgtagtattttccgcatgct

SEQ ID NO: 39 - DNA, 18 bp, Homo sapiens
ggctggtgacaggccaaa

SEQ ID NO: 40 - DNA, 21 bp, Homo sapiens
cagtgccacagtggactctga

SEQ ID NO: 41 - DNA, 21 bp, Homo sapiens
gctggactccatcaccacact

SEQ ID NO: 42 - DNA, 23 bp, Homo sapiens
agttggtcaaaaggtccttcatg

SEQ ID NO: 43 - DNA, 20 bp, Homo sapiens
gaagcgtgcacaccaacact

SEQ ID NO: 44 - DNA, 22 bp, Homo sapiens
cctcttgagcatcttcggaatt

SEQ ID NO: 45 - DNA, 23 bp, Homo sapiens
gatcaccttatggatgtcgcaat

SEQ ID NO: 46 - DNA, 23 bp, Homo sapiens
accccagctctcttgatgagaag

SEQ ID NO: 47 - DNA, 19 bp, Homo sapiens
ctcagcctgctccgtgatg

SEQ ID NO: 48 - DNA, 22 bp, Homo sapiens
gcacgtgtgtacatgtctgcat

SEQ ID NO: 49 - DNA, 25 bp, Homo sapiens
ctcgtctgtcttgagttgtgacatt

SEQ ID NO: 50 - DNA, 23 bp, Homo sapiens
tcttgtcaccagagcctgttaca

SEQ ID NO: 51 - DNA, 21 bp, Homo sapiens
tccctttgaggtgtcctacca

SEQ ID NO: 52 - DNA, 24 bp, Homo sapiens
gtcatccaggtacagcactttcac

SEQ ID NO: 53 - DNA, 23 bp, Homo sapiens
tcagggaacagttttgccttaag

SEQ ID NO: 54 - DNA, 23 bp, Homo sapiens
ccctcagaagtattccctccatt

SEQ ID NO: 55 - DNA, 21 bp, Homo sapiens
cgctctcaactggagcaaact

SEQ ID NO: 56 - DNA, 22 bp, Homo sapiens
gccctcagggaaaaacctagtg

SEQ ID NO: 57 - DNA, 24 bp, Homo sapiens
tcttagtggtttgtggaattgggt

SEQ ID NO: 58 - DNA, 21 bp, Homo sapiens
cggtaaggtaaatcgtgtggc

SEQ ID NO: 59 - DNA, 20 bp, Homo sapiens
cctgcttggaaaccatgtca

SEQ ID NO: 60 - DNA, 22 bp, Homo sapiens
gggtggtattggtctgctgaag

SEQ ID NO: 61 - DNA, 18 bp, Homo sapiens
accccagaggtgaaggcc

SEQ ID NO: 62 - DNA, 22 bp, Homo sapiens
tggtttcggaggtcactcatct

SEQ ID NO: 63 - DNA, 24 bp, Homo sapiens
tttactcaggagccacatgatgtc

SEQ ID NO: 64 - DNA, 24 bp, Homo sapiens
ggtaaccattagcagcacaattca

SEQ ID NO: 65 - DNA, 24 bp, Homo sapiens
aatgatgaagaggaacatggcata

SEQ ID NO: 66 - DNA, 26 bp, Homo sapiens
tggtttccaggtctttaatgtagtcc

SEQ ID NO: 67 - DNA, 20 bp, Homo sapiens
aaattgagacagggcctgca

SEQ ID NO: 68 - DNA, 27 bp, Homo sapiens
cagtattctcattttcattgttccatc

SEQ ID NO: 69 - DNA, 23 bp, Homo sapiens
acccccttccatcaaatctctag

SEQ ID NO: 70 - DNA, 18 bp, Homo sapiens
gccgccttcgcttacaag

SEQ ID NO: 71 - DNA, 25 bp, Homo sapiens
cagtgatagagaattgctgtggaga

SEQ ID NO: 72 - DNA, 21 bp, Homo sapiens
ctttacacactggcaggcctc

SEQ ID NO: 73 - DNA, 24 bp, Homo sapiens
aacctaaccttccatatgcacacc

SEQ ID NO: 74 - DNA, 19 bp, Homo sapiens
aaaccctttgccgcaagtg

SEQ ID NO: 75 - DNA, 25 bp, Homo sapiens
ttctggaatccaaaccacagtctta

SEQ ID NO: 76 - DNA, 24 bp, Homo sapiens
ctgctgttcttctatggtgcctaa

SEQ ID NO: 77 - DNA, 20 bp, Homo sapiens
accccgtctccgctaaaaat

SEQ ID NO: 78 - DNA, 19 bp, Homo sapiens
cctgccgagtagctgggac

SEQ ID NO: 79 - DNA, 25 bp, Homo sapiens
atgcaagtgtgaattacgtggtatg

SEQ ID NO: 80 - DNA, 21 bp, Homo sapiens
acgggcagtttgctgtacatc

SEQ ID NO: 81 - DNA, 24 bp, Homo sapiens
tgtcgagcattctctttctctgtt

SEQ ID NO: 82 - DNA, 22 bp, Homo sapiens
cccctttctgtaggattccctt

SEQ ID NO: 83 - DNA, 22 bp, Homo sapiens
ctttggtgcctttcctgttcag

SEQ ID NO: 84 - DNA, 25 bp, Homo sapiens
tgtcgtcatgtagcacaaagtttct

SEQ ID NO: 85 - DNA, 28 bp, Homo sapiens
gagtgactaaaggaatggaagttgtaca

SEQ ID NO: 86 - DNA, 22 bp, Homo sapiens
gggcttatctgttttgggattg

SEQ ID NO: 87 - DNA, 23 bp, Homo sapiens
atgggatacagctggtcaagaac

SEQ ID NO: 88 - DNA, 19 bp, Homo sapiens
tggctgcttgtgctcctct

SEQ ID NO: 89 - DNA, 22 bp, Homo sapiens
catcaccgtctactgcgtctgt

SEQ ID NO: 90 - DNA, 19 bp, Homo sapiens
gctccttctccttgcgctg

SEQ ID NO: 91 - DNA, 22 bp, Homo sapiens
ctgcactcaatgctggaatcaa

SEQ ID NO: 92 - DNA, 28 bp, Homo sapiens
agatttctttcctgtttacatcttgttg

SEQ ID NO: 93 - DNA, 25 bp, Homo sapiens
gctacagagagatcccaagagaagg

SEQ ID NO: 94 - DNA, 19 bp, Homo sapiens
ggtccactccctgaagcca

SEQ ID NO: 95 - DNA, 19 bp, Homo sapiens
gagacgaggctaccgctgc

SEQ ID NO: 96 - DNA, 25 bp, Homo sapiens
cacttttatttcctgtttgggagaa

SEQ ID NO: 97 - DNA, 21 bp, Homo sapiens
cagcctgaaggaaatacgcaa

SEQ ID NO: 98 - DNA, 19 bp, Homo sapiens
tgagccagaggaaaaggcc

SEQ ID NO: 99 - DNA, 21 bp, Homo sapiens
agcaacttttgcttggcaaaa

SEQ ID NO: 100 - DNA, 21 bp, Homo sapiens
cacctcctccttcctctgctg

SEQ ID NO: 101 - DNA, 19 bp, Homo sapiens
tcctgaattccccttgcca

SEQ ID NO: 102 - DNA, 23 bp, Homo sapiens
agatgatacatgagatggaggcc

SEQ ID NO: 103 - DNA, 23 bp, Homo sapiens
gcatctgggaatacagggtagaa

SEQ ID NO: 104 - DNA, 22 bp, Homo sapiens
gggagacagtggcaatgtgtag

SEQ ID NO: 105 - DNA, 20 bp, Homo sapiens
cagcctgtcccatggacact

SEQ ID NO: 106 - DNA, 19 bp, Homo sapiens
tcctctggcccctgagatg

SEQ ID NO: 107 - DNA, 20 bp, Homo sapiens
ttcaaggctgggaggaacat

SEQ ID NO: 108 - DNA, 20 bp, Homo sapiens
tgacacttcagccgcaagac

SEQ ID NO: 109 - DNA, 22 bp, Homo sapiens
ctcgtgaagagcagcacaacat

SEQ ID NO: 110 - DNA, 20 bp, Homo sapiens
ctgtgctccattccctcaca

SEQ ID NO: 111 - DNA, 21 bp, Homo sapiens
tgcaagactgggaatggaaag

SEQ ID NO: 112 - DNA, 23 bp, Homo sapiens
caggtgatgccattttttgtttt

SEQ ID NO: 113 - DNA, 19 bp, Homo sapiens
cgtgctggtgcaggtcagt

SEQ ID NO: 114 - DNA, 22 bp, Homo sapiens
catacagctttacccggcaaat

SEQ ID NO: 115 - DNA, 23 bp, Homo sapiens
ttcttatttgccagagccaaatt

SEQ ID NO: 116 - DNA, 32 bp, Homo sapiens
tccagacaatatgtagttaacatttaggctaa